SEQUENCE LISTING (53 sequences)

SEQ ID NO: 1 - protein, 112 aa, Artificial sequence
  anti-RAS intracellular antibody
QVQLVESGGGLVQPGGSLRLSCAASGFTFSSYAMHWVRQAPGKGLEWVSVISGDGSNTYYADSVKGRFTISRDNSKNTLYLQMNSLRAEDTAVYYCARGDYWGQGTLVTVSS

SEQ ID NO: 2 - protein, 115 aa, Artificial sequence
  anti-RAS intracellular antibody
QVQLVEAGGGLVQPGGSLRLSCAASGFSFSYLYMNWVRQAPGKGLEWVSYISSSGRTIYYADSVKGRFTISRDNAKNSLYLQMNSLRDEDTAVYYCARGRFFDYWGQGTLVTVSS

SEQ ID NO: 3 - protein, 115 aa, Artificial sequence
  anti-RAS intracellular antibody
QVQLVEAGGGLVQPGGSLRLSCAASGFSFSTFSMNWVRQAPGKGLEWVSYISSSGRTIYYADSVKGRFTISRDNAKNSLYLQMNSLRDEDTAVYYCARGRFFDYWGQGTLVTVSS

SEQ ID NO: 4 - protein, 116 aa, Artificial sequence
  anti-RAS intracellular antibody
EVQLLESGGGLVQPGGSLRLSCAASGFTFSSYAMSWVRQAPGKGLEWVSTISYGGSNTNYADSVKARFTISRDNSKNTLYLQMNSLRAEDTAVYYCAKGNNAFDYWGQGTLVTVSS

SEQ ID NO: 5 - protein, 126 aa, Artificial sequence
  anti-RAS intracellular antibody
EVQLVESGGGLVQPGGSLRLSCAASGFTFSSYAMSWVRQAPGKGLEWVSVISGKTDSTYYADSVKGRFTISRDNSKNTLYLQMNSLRAEDTAVYYCARGRGSLSGYYYYHYPFDYWGQGTLVTVSS

SEQ ID NO: 6 - protein, 115 aa, Artificial sequence
  anti-RAS intracellular antibody
EVQLLESGGGLVQPGGSLRLSCAASGFSFSYLYMNWVRQAPGKGLEWVSYISSSGRTIYYADSVKGRFTISRDNSKNTLYLQMNSLRAEDTAVYYCAKGRFFDYWGQGTLVTVSS

SEQ ID NO: 7 - protein, 115 aa, Artificial sequence
  anti-RAS intracellular antibody
EVQLLESGGGLVQPGGSLRLSCAASGFSFSYLYMNWVRQAPGKGLEWVSYISSSGRTIYYADSVKGRFTISRDNSKNTLYLQMNSLRAEDTAVYYCARGRFFDYWGQGTLVTVSS

SEQ ID NO: 8 - protein, 115 aa, Artificial sequence; identical to SEQ ID NO: 7
  anti-RAS intracellular antibody

SEQ ID NO: 9 - protein, 115 aa, Artificial sequence
  anti-RAS intracellular antibody
QVQLVESGGGLVQPGGSLRLSCAASGFSFSYLYMNWVRQAPGKGLEWVSYISSSGRTIYYADSVKGRFTISRDNSKNTLYLQMNSLRAEDTAVYYCARGRFFDYWGQGTLVTVSS

SEQ ID NO: 10 - protein, 115 aa, Artificial sequence
  anti-RAS intracellular antibody
EVQLLESGGGLVQPGGSLRLSSAASGFSFSYLYMNWVRQAPGKGLEWVSYISSSGRTIYYADSVKGRFTISRDNSKNTLYLQMNSLRAEDTAVYYSARGRFFDYWGQGTLVTVSS

SEQ ID NO: 11 - protein, 108 aa, Artificial sequence
  anti-RAS intracellular antibody
DIVMTQSPSSLSASVGDRVTITCRASQSISYYLNWYQQKPGKAPKLLIYAASTLQSGVPSRFSGSGSGTDFTLTISSLQPEDFATYYCQQYYSTPRTFGQGTKVEIKR

SEQ ID NO: 12 - protein, 108 aa, Artificial sequence
  anti-RAS intracellular antibody
DIVMTQSPSFLSASVGDRVTITCRASQSISRYLNWYQQKPGKAPKLLIYAASSLQSGVPSRFSGSGSGTDFTLTISSLQPEDFAIYYCQQSYSTLLTFGGGTKLEIKR

SEQ ID NO: 13 - protein, 108 aa, Artificial sequence; identical to SEQ ID NO: 12
  anti-RAS intracellular antibody

SEQ ID NO: 14 - protein, 109 aa, Artificial sequence
  anti-RAS intracellular antibody
TDIQMTQSPSSLSASVGDRVTITCRASQSISSYLNWYQQKPGKAPKLLIYNASALQSGVPSRFSGSGSGTDFTLTISSLQPEDFATYYCQQYTSTPRTFGQGTKVEIKR

SEQ ID NO: 15 - protein, 112 aa, Artificial sequence
  anti-RAS intracellular antibody
DIQMTQSPSSLSASVGDRVTITCRASQSLVSISNYLAWYQQKPGKAPKLLIYAASSLESGVPSRFSGSGSGTDFTLTISSLQPEDFATYYCQQYNSLPQWTFGQGTKVEIKR

SEQ ID NO: 16 - protein, 109 aa, Artificial sequence
  anti-RAS intracellular antibody
TDIQMTQSPSSLSASVGDRVTITCRASQSISRYLNWYQQKPGKAPKLLIYAASSLQSGVPSRFSGSGSGTDFTLTISSLQPEDFATYYCQQSYSTLLTFGQGTKVEIKR

SEQ ID NO: 17 - protein, 109 aa, Artificial sequence; identical to SEQ ID NO: 16
  anti-RAS intracellular antibody

SEQ ID NO: 18 - protein, 109 aa, Artificial sequence; identical to SEQ ID NO: 14
  anti-RAS intracellular antibody

SEQ ID NO: 19 - protein, 108 aa, Artificial sequence
  anti-RAS intracellular antibody
DIVMTQSPSSLSASVGDRVTITCRASQSISSYLNWYQQKPGKAPKLLIYNASALQSGVPSRFSGSGSGTDFTLTISSLQPEDFATYYCQQYTSTPRTFGQGTKVEIKR

SEQ ID NO: 20 - protein, 109 aa, Artificial sequence; identical to SEQ ID NO: 16
  anti-RAS intracellular antibody

SEQ ID NO: 21 - protein, 125 aa, Artificial sequence
  anti-RAS intracellular antibody
QVQLVESGGGLVQPGGSLRLSCAASGFTFSSYAMHWVRQAPGKGLEWVSVISKLTHHTYYADSVKGRFTISRDNSKNTLYLQMNSLRAEDTAVYYCARGVQGYVHGLKGNWFDYWGQGTLVTVSS

SEQ ID NO: 22 - protein, 115 aa, Artificial sequence
  anti-RAS intracellular antibody
EVQLLESGGGLVQPGGSLRLSCAASGFSFSTFSMNWVRQAPGKGLEWVSYISRTSKTIYYADSVKGRFTISRDNSKNTLYLQMNSLRAEDTAVYYCARGRFFDYWGQGTLVTVSS

SEQ ID NO: 23 - protein, 115 aa, Artificial sequence
  anti-RAS intracellular antibody
EVQLLESGGGLVQPGGSLRLSCAASGFSFSTFSMNWVRQAPGKGLEWVSYISATARSIYYADSVKGRFTISRDNSKNTLYLQMNSLRAEDTAVYYCARGGRFDYWGQGTLVTVSS

SEQ ID NO: 24 - protein, 115 aa, Artificial sequence
  anti-RAS intracellular antibody
EVQLLESGGGLVQPGGSLRLSCAASGFSFSTFSMNWVRQAPGKGLEWVSYISTSGRTIYYADSVKGRFTISRDNSKNTLYLQMNSLRAEDTAVYYCARGQKFDYWGQGTLVTVSS

SEQ ID NO: 25 - protein, 115 aa, Artificial sequence
  anti-RAS intracellular antibody
EVQLLESGGGLVQPGGSLRLSCAASGFSFSVWAMSWVRQAPGKGLEWVSYISRTSKTIYYADSVKGRFTISRDNSKNTLYLQMNSLRAEDTAVYYCARGRFFDYWGQGTLVTVSS

SEQ ID NO: 26 - protein, 115 aa, Artificial sequence
  anti-RAS intracellular antibody
EVQLLESGGGLVQPGGSLRLSCAASGFSFSTFSMNWVRQAPGKGLEWVSYISRTSMAIYYADSVKGRFTISRDNSKNTLYLQMNSLRAEDTAVYYCARGRFFDYWGQGTLVTVSS

SEQ ID NO: 27 - protein, 115 aa, Artificial sequence
  anti-RAS intracellular antibody
EVQLLESGGGLVQPGGSLRLSCAASGFSFSTFSMNWVRQAPGKGLEWVSYISSSRHSIYYADSVKGRFTISRDNSKNTLYLQMNSLRAEDTAVYYCARGSRFDYWGQGTLVTVSS

SEQ ID NO: 28 - protein, 115 aa, Artificial sequence
  anti-RAS intracellular antibody
EVQLLESGGGLVQPGGSLRLSCAASGFSFSTFSMNWVRQAPGKGLEWVSYISCTSHCIYYADSVKGRFTISRDNSKNTLYLQMNSLRAEDTAVYYCARGRFFDYWGQGTLVTVSS

SEQ ID NO: 29 - protein, 117 aa, Artificial sequence
  anti-RAS intracellular antibody
QVQLVESGGGLVQPGGSLRLSCAASGFTFSSYAMHWVRQAPGKGLEWVSVISSFNWQTYYADSVKGRFTISRDNSKNTLYLQMNSLRAEDTAVYYCARGSGQSFDYWGQGTLVTVSS

SEQ ID NO: 30 - protein, 117 aa, Artificial sequence; identical to SEQ ID NO: 29
  anti-RAS intracellular antibody

SEQ ID NO: 31 - protein, 119 aa, Artificial sequence
  anti-RAS intracellular antibody
QVQLVESGGGLVQPGGSLRLSCAASGFTFSSYAMHWVRQAPGKGLEWVSVISSFNFTTYYADSVKGRFTISRDNSKNTLYLQMNSLRAEDTAVYYCARGSTGMLSFDYWGQGTLVTVSS

SEQ ID NO: 32 - protein, 117 aa, Artificial sequence
  anti-RAS intracellular antibody
QVQLVESGGGLVQPGGSLRLSCAASGFTFSSYAMHWVRQAPGKGLEWVSVISSFNHNTYYADSVKGRFTISRDNSKNTLYLQMNSLRAEDTAVYYCARGTHESFDYWGQGTLVTVSS

SEQ ID NO: 33 - protein, 115 aa, Artificial sequence
  anti-RAS intracellular antibody
QVQLVESGGGLVQPGGSLRLSCAASGFTFSSYAMHWVRQAPGKGLEWVSVISMMNHNTYYADSVKGRFTISRDNSKNTLYLQMNSLRAEDTAVYYCARGRPFDYWGQGTLVTVSS

SEQ ID NO: 34 - protein, 117 aa, Artificial sequence
  anti-RAS intracellular antibody
QVQLVESGGGLVQPGGSLRLSCAASGFTFSSYAMHWVRQAPGKGLEWVSVISAFNWNTYYADSVKGRFTISRDNSKNTLYLQMNSLRAEDTAVYYCARGLEIGFDYWGQGTLVTVSS

SEQ ID NO: 35 - protein, 115 aa, Artificial sequence; identical to SEQ ID NO: 26
  anti-RAS intracellular antibody

SEQ ID NO: 36 - protein, 117 aa, Artificial sequence
  anti-RAS intracellular antibody
QVQLVESGGGLVQPGGSLRLSCAASGFTFSSYAMHWVRQAPGKGLEWVSVISAFNWNTYYADSVKGRFTISRDNSKNTLYLQMNSLRAEDTAVYYCARGSGQSFDYWGQGTLVTVSS

SEQ ID NO: 37 - protein, 117 aa, Artificial sequence; identical to SEQ ID NO: 29
  anti-RAS intracellular antibody

SEQ ID NO: 38 - protein, 115 aa, Artificial sequence
  anti-RAS intracellular antibody
QVQLVESGGGLVQPGGSLRLSCAASGFTFSSYAMHWVRQAPGKGLEWVSVISAFNWNTYYADSVKGRFTISRDNSKNTLYLQMNSLRAEDTAVYYCARGNQFDYWGQGTLVTVSS

SEQ ID NO: 39 - protein, 123 aa, Artificial sequence
  anti-RAS intracellular antibody
EVQLLESGGGLVQPGGSLRLSCAASGFSFSTFSMNWVRQAPGKGLEWVSYISAAATEIYYADSVKGRFTISRDNSKNTLYLQMNSLRAEDTAVYYCARGPRHHQLGWMVFDYWGQGTLVTVSS

SEQ ID NO: 40 - protein, 123 aa, Artificial sequence
  anti-RAS intracellular antibody
QVQLVESGGGLVQPGGSLRLSCAASGFTFSSYAMSWVRQAPGKGLEWVSTISYGGSNTNYADSVKGRFTISRDNSKNTLYLQMNSLRAEDTAVYYCARGRALQDANYLLFDYWGQGTLVTVSS

SEQ ID NO: 41 - protein, 119 aa, Artificial sequence
  anti-RAS intracellular antibody
QVQLVESGGGLVQPGGSLRLSCAASGFTFSSYAMHWVRQAPGKGLEWVSVISGASQVTYYADSVKGRFTISRDNSKNTLYLQMNSLRAEDTAVYYCARGNRDVGMFDYWGQGTLVTVSS

SEQ ID NO: 42 - protein, 117 aa, Artificial sequence
  anti-RAS intracellular antibody
EVQLLESGGGLVQPGGSLRLSCAASGFSFSTFSMNWVRQAPGKGLEWVSYISRYGTRIYYADSVKGRFTISRDNSKNTLYLQMNSLRAEDTAVYYCARGGVSTFDYWGQGTLVTVSS

SEQ ID NO: 43 - protein, 125 aa, Artificial sequence
  anti-RAS intracellular antibody
EVQLLESGGGLVQPGGSLRLSCAASGFSFSTFSMNWVRQAPGKGLEWVSYISSSGTRIYYADSVKGRFTISRDNSKNTLYLQMNSLRAEDTAVYYCARGQRWPPTPGPFTLLDYWGQGTLVTVSS

SEQ ID NO: 44 - protein, 117 aa, Artificial sequence
  anti-RAS intracellular antibody
QVQLVESGGGLVQPGGSLRLSCAASGFTFSSYAMHWVRQAPGKGLEWVSVISRSGKITYYADSVKGRFTISRDNSKNTLYLQMNSLRAEDTAVYYCARGGVTNFDYWGQGTLVTVSS

SEQ ID NO: 45 - protein, 123 aa, Artificial sequence
  anti-RAS intracellular antibody
EVQLLESGGGLVQPGGSLRLSCAASGFSFSTFSMNWVRQAPGKGLEWVSYISGTGSQIYYADSVKGRFTISRDNSKNTLYLQMNSLRAEDTAVYYCARGEWTMLREQLLFDYWGQGTLVTVSS

SEQ ID NO: 46 - protein, 125 aa, Artificial sequence
  anti-RAS intracellular antibody
EVQLLESGGGLVQPGGSLRLSCAASGFSFSTFSMNWVRQAPGKGLEWVSYISSAGGQIYYADSVKGRFTISRDNSKNTLYLQMNSLRAEDTAVYYCARGACDRLTCLRTYAFDYWGQGTLVTVSS

SEQ ID NO: 47 - protein, 123 aa, Artificial sequence; identical to SEQ ID NO: 45
  anti-RAS intracellular antibody

SEQ ID NO: 48 - protein, 123 aa, Artificial sequence
  anti-RAS intracellular antibody
EVQLLESGGGLVQPGGSLRLSCAASGFSFSTFSMNWVRQAPGKGLEWVSYISKHGSSIYYADSVKGRFTISRDNSKNTLYLQMNSLRAEDTAVYYCARGYVSVTSSWAFFDYWGQGTLVTVSS

SEQ ID NO: 49 - protein, 15 aa, Artificial sequence
  Amino acid linker sequence
GGGGSGGGGSGGGGS

SEQ ID NO: 50 - protein, 9 aa, Human immunodeficiency virus type 1
RKKRRQRRR

SEQ ID NO: 51 - protein, 15 aa, Human immunodeficiency virus type 1
CGRKKRRQRRRPPQC

SEQ ID NO: 52 - protein, 16 aa, Drosophila melanogaster
RQIKIWFQNRRMKWKK

SEQ ID NO: 53 - protein, 9 aa, Artificial sequence
  FLAG-tag peptide
MDYKDDDDK

(19) United States
(12) Patent Application Publication
(10) Pub. No.: US 2005/0288492 A1
(43) Pub. Date: Dec. 29, 2005

(54) Anti-activated RAS antibodies

(75) Inventors: Terrence Howard Rabbitts, Cambridge (GB); Tomoyuki Tanaka, Cambridge (GB)

Correspondence Address: PALMER & DODGE, LLP;KATHLEEN M. WILLIAMS, 111 HUNTINGTON AVENUE, BOSTON, MA, 02199, US

(73) Assignee: Medical Research Council

(21) Appl. No.: 11/127,903

(22) Filed: May 12, 2005

Related U.S. Application Data
(63) Continuation of application No. PCT/GB03/04953, filed on Nov. 14, 2003.

(30) Foreign Application Priority Data
Nov. 15, 2002 (GB) 0226728.4
Nov. 15, 2002 (GB) 0226729.2
Nov. 15, 2002 (GB) 0226723.5
Nov. 15, 2002 (GB) 0226731.8
Nov. 15, 2002 (GB) 0226727.6
Jul. 16, 2003 (GB) 0316680.8

(51) Int. Cl. C07K 16/40; C07H 21/04; C12P 21/06; C12N 15/74; C12N 5/06

(52) U.S. Cl. 530/388.26; 435/69.1; 435/320.1; 435/330; 536/23.53

(57) ABSTRACT

The present invention relates to antibodies that function within an intracellular environment. In particular the present invention related to a particular antibodies which the inventors have shown to bind to the activated form of RAS. Uses of such an antibody are also described. Anti-activated RAS antibodies The present invention relates to antibodies that function within an intracellular environment. In particular the present invention relates to a particular antibodies which the inventors have shown to bind to the activated form of RAS. Uses of such an antibody are also described.

The present invention relates to antibodies that function within an intracellular environment. In particular the present invention relates to a particular antibodies which the inventors have shown to bind to the activated form of RAS. Uses of such an antibody are also described.

Intracellular antibodies or intrabodies have been demonstrated to function in antigen recognition in the cells of higher organisms (reviewed in Cattaneo, A. & Biocca, S. (1997) Intracellular Antibodies: Development and Applications. Landes and Springer-Verlag). This interaction can influence the function of cellular proteins which have been successfully inhibited in the cytoplasm, the nucleus or in the secretory pathway. This efficacy has been demonstrated for viral resistance in plant biotechnology (Tavladoraki, P., et al. (1993) Nature 366: 469-472) and several applications have been reported of intracellular antibodies binding to HIV viral proteins (Mhashilkar, A. M., et al. (1995) EMBO J 14: 1542-51; Duan, L. & Pomerantz, R. J. (1994) Nucleic Acids Res 22: 5433-8; Maciejewski, J. P., et al. (1995) Nat Med 1: 667-73; Levy-Mintz, P., et al. (1996) J. Virol. 70: 8821-8832) and to oncogene products (Biocca, S., Pierandrei-Amaldi, P. & Cattaneo, A. (1993) Biochem Biophys Res Commun 197: 422-7; Biocca, S., Pierandrei-Amaldi, P., Campioni, N. & Cattaneo, A. (1994) Biotechnology (N Y) 12: 396-9; Cochet, O., et al. (1998) Cancer Res 58: 1170-6). The latter is an important area because enforced expression of oncogenes often occurs in tumour cells after chromosomal translocations (Rabbitts, T. H. (1994) Nature 372: 143-149). These proteins are therefore important intracellular therapeutic targets (Rabbitts, T. H. (1998) New Eng. J. Med 338: 192-194) which could be inactivated by binding with intracellular antibodies. Finally, the international efforts at whole genome sequencing will produce massive numbers of potential gene sequences which encode proteins about which nothing is known. Functional genomics is an approach to ascertain the function of this plethora of proteins and the use of intracellular antibodies promises to be an important tool in this endeavour as a conceptually simple approach to knocking-out protein function directly by binding an antibody inside the cell.

Simple approaches to derivation of antibodies which function in cells are therefore necessary if their use is to have any impact on the large number of protein targets. In normal circumstances, the biosynthesis of immunoglobulin occurs into the endoplasmic reticulum for secretion as antibody. However, when antibodies are expressed in the cell cytoplasm (where the redox conditions are unlike those found in the ER) folding and stability problems occur resulting in low expression levels and the limited half-life of antibody domains. These problems are most likely due to the reducing environment of the cell cytoplasm (Hwang, C., Sinskey, A. J. & Lodish, H. F. (1992) Science 257: 1496-502), which hinders the formation of the intrachain disulphide bond of the VH and VL domains (Biocca, S., Ruberti, F., Tafani, M., Pierandrei-Amaldi, P. & Cattaneo, A. (1995) Biotechnology (N Y) 13: 1110-5; Martineau, P., Jones, P. & Winter, G. (1998) J Mol Biol 280: 117-127) important for the stability of the folded protein. However, some scFv have been shown to tolerate the absence of this bond (Proba, K., Honegger, A. & Pluckthun, A. (1997) J Mol Biol 265: 161-72; Proba, K., Worn, A., Honegger, A. & Pluckthun, A. (1998) J Mol Biol 275: 245-53) which presumably depends on the particular primary sequence of the antibody variable regions. No rules or consistent predictions until the present invention, been made about those antibodies which will tolerate the cell cytoplasm conditions. A further problem is the design of expression formats for intracellular antibodies and much effort has be expended on using scFv in which the VH and VL segments (i.e. the antibody combining site) are linked by a polypeptide linker at the C-terminus of VH and the N-terminus of V_{L} (Bird, R. E., et al. (1988) Science 242: 423-6). While this is the most successful form for intracellular expression, it has a drawback in the lowering of affinity when converting from complete antibody (e.g. from a monoclonal antibody) to a scFv. Thus not all monoclonal antibodies can be made as scFv and maintain function in cells. Finally, different scFv fragments have distinct properties of solubility or propensity to aggregate when expressed in this cellular environment.

Antibodies are used extensively in bioscience as in vitro tools for recognising target antigens and for medical applications such as diagnosis or therapeutics. Recently gene cloning technologies have allowed the genes for coding antibodies to be manipulated and expressed intracellularly (Cattaneo and Biocca, 1999a). Intracellular antibodies (ICAb) with specific and high-affinity binding properties have great potential for application in the therapy of human diseases in which target proteins or protein interactions are found only inside the target cell. A suitable form for ICAb expression is the single-chain antibody, also known as single chain variable fragment or scFv (Biocca et al., 1994; Cohen, 2002; Marasco et al., 1993), which is composed of the heavy and light-chain variable domains and a flexible linker peptide to fuse them (Bird et al., 1988; Huston et al., 1988).

Application of functional scFv as ICAbs have been exploited and achieved in several fields. There is potential for their use in cancer cells, where there occurs chromosomal translocations or somatic mutations effectively producing tumour-specific intracellular proteins (Rabbitts, 1994; Rabbitts and Stocks, 2002). As the protein products are inside in the cell, rather than exposed on the cell surface, conventional antibody therapy is not an option. The scFv format is suitable for intracellular use because of its optimal size and ease of expression from vectors since the VH and VL segments are present on a single macromolecule, and thus requiring no inter-chain disulphide linkage to hold together the two chains. Several such antibody fragments have been demonstrated to be effective in targeting proteins in vivo (Biocca et al., 1993; Rondon and Marasco, 1997; Tavladoraki et al., 1993), but there remain few antibodies which work effectively in intracellular reducing environment because there are often problems with correct folding and their resulting in lack of function, low expression and short half life (Cattaneo and Biocca, 1999b). Indeed, it has been generally found that most of scFv which are derived from hybridomas do not function effectively in vivo, regardless of their having sufficient high affinity and antigen specificity. Furthermore, the intra-domain disulphide bond does not form in scFv expressed in the cytoplasm of eukaryotic cells bonds (Biocca et al., 1995) but some scFv have been shown to tolerate the absence of this bond (Proba et al., 1998; Worn and Pluckthun, 1998a). At this time, there is no general rule or prediction of the requirements for soluble and stable intracellular antibodies.

In this regard, several approaches have been adopted to solve this problem. These include the modification of the sequence of VH and VL domains utilising random mutation to replace the need for disulphide bonds to stabilise scFv with high intrinsic stability (Proba et al., 1998; Worn and Pluckthun, 1998b) or use of frameworks which empirically prove to be effective in vivo (Tse et al., 2002; Visintin et al., 2002).

However at the time of filing, there remains a need in the art to identify characteristics of intracellular antibodies which allow them to bind to the oncogenic form of RAS within an intracellular environment. Such antibodies will have wide ranging propylactic and therapeutic applications.

### SUMMARY OF THE INVENTION

The present inventors recently developed a selection method to isolate intracellular antibodies which primarily depends on their function inside yeast and mammalian cells, described as intracellular antibody capture (IAC) technology (Visintin et al., 1999) (WO00/54057).

Using the IAC approach, the present inventors have now identified three anti-RAS antibodies which are capable of binding specifically to RAS within an intracellular environment. These antibodies exhibit different characteristics with regard to their in vivo antigen affinity, solubility and stability. In addition the inventors have shown that antibodies comprising either light or heavy chain variable domains, but not both, are capable of specifically binding to activated RAS. In addition, the present inventors have modified the IAC approach to exclude the initial in vitro selection method. They have called this approach the IAC² approach. In particular the inventors used a previously characterised intrabody single variable domain (IDab) format, based on a previously characterised consensus scaffold, to generate diverse intrabody libraires for direct in vivo screening. In this way a further panel of anti-RAS specific intracellular antibodies was isolated.

The inventors have surprisingly found that the formation of a heavy variable domain intradomain disulphide bridge is not required in order to obtain an antibody which binds specifically within an intracellular environment. The antibodies described herein are specific for the mutant/activated form of RAS but not the native/non-activated form of RAS. The inventors believe that such antibodies are of considerable prophylactic and therapeutic use.

Thus, in a first aspect, the present invention provides an antibody molecule capable of specifically binding to activated RAS within an intracellular environment wherein the antibody comprises a single variable domain type only (single domain type antibody) and such variable domain comprises any of the amino acid sequences selected from the groups consisting of:
(a) in the case of VH: Con, J48, 33, I21R33, I21R33VHI21VL, Con 33 and I21R33(VHC22S;C92S) as depicted in FIG. 3 and designated SEQ 1, SEQ 2, SEQ 3, SEQ 7, SEQ 8, SEQ 9, SEQ 10 respectively, or any of the sequences listed above in which one or more of residues 22 and 92 are not cysteine residuesSEQ No 21, SEQ No 22, SEQ No 23, SEQ No 24, SEQ No 25, SEQ No 26, SEQ No 27, SEQ No 28 and SEQ No 29 as depicted in FIG. 3; and
(b) in the case of VL: Con, J48, 33, 12IR33, I21R33VHI21VL, Con 33 and I21R33(VHC22S;C92S) as depicted in FIG. 3 and designated for VL: SEQ 11, SEQ 12, SEQ 13, SEQ 17, SEQ 18, SEQ 19, SEQ 20.

As referred to herein the term ‘a single variable domain type antibody’ means an antibody as herein defined which comprises either one or more heavy chain variable domains or one or more light chain domains but not both heavy and light chain variable domains. Advantageously, a single variable domain type antibody according to the invention is a Dab (IDab). As herein defined a ‘Dab’ is a single variable heavy chain domain or a single variable light chain domain optionally attached to a ‘bulking group’. The ‘bulking group’ as herein defined may comprise one or more antibody constant region domains. Alternatively, the ‘bulking group’ may comprise components of non-immunoglobulin origin. These may include cytotoxins, fluorescent or other forms of labels. Those skilled in the art will appreciate that this list is not intended to be exhaustive. For the avoidance of any doubt a Dab (IDab) according to the invention may comprise only a light or heavy chain variable domain. Most advantageously, a ‘Dab’ according to the present invention comprises a single heavy chain variable domain.

In a further aspect still the present invention provides an antibody molecule capable of specifically binding to activated RAS within an intracellular environment wherein the antibody comprises a heavy chain variable domain and a light chain variable domain wherein the heavy chain variable domain and the light chain variable domain of the antibody comprise any of the amino acid sequences selected from the group consisting of: Con, J48, 33, I21R33, I21R33VHI21VL, Con 33 and I21R33(VHC22S;C92S) as depicted in FIG. 3 and designated SEQ 1, SEQ 2, SEQ 3, SEQ 7, SEQ 8, SEQ 9 and SEQ 10 respectively in the case of variable heavy chain domains or any of the sequences listed above in which one or more of residues 22 and 92 (according to Kabat numbering) are not cysteine residues and the corresponding light chain domains as depicted in FIG. 3.

According to the above aspect of the invention, the term ‘corresponding light chain’ refers to that light chain which is paired with a particular heavy chain within the same scFv molecule identified in FIG. 3. That is, the corresponding light chain of the J48 heavy chain sequence is the J48 light chain sequence. Moreover, the corresponding light chain of the 33 heavy chain sequence is the 33 light chain sequence.

According to the above aspects of the invention, the term ‘specific binding to activated RAS’ means that within a mixture of reagents comprising activated RAS in addition to other alternative antigens, only activated RAS is bound. That is, the binding of an anti-RAS antibody according to the present invention is selective for activated RAS.

The specific binding of an antibody according to the present invention to activated RAS may be of high affinity or low affinity. For example, the specific interaction of scFv I21 with activated RAS is of low affinity whereas the specific interaction of scFv 33 with activated RAS is of high affinity. Affinity measurements may be made using methods known to those skilled in the art including using BIACORE measurements as herein described.

According to the above aspect of the invention, preferably the antibody is an scFv or a Dab as herein defined. Most preferably the antibody is an scFv which specifically binds as herein defined activated RAS.

In a further aspect still the present invention provides an antibody molecule for functionally inactivating activated RAS within an intracellular environment wherein the antibody comprises a single variable domain type only and such variable domain comprises any of the amino acid sequences selected from the groups consisting of:
(a) in the case of VH: Con, J48, 33, I21R33, 21R33VHI21VL, Con 33 and I21R33(VHC22S;C92S) as depicted in FIG. 3 and designated SEQ 1, SEQ 2, SEQ 3, SEQ 7, SEQ 8, SEQ 9, SEQ 10, respectively; or any of the sequences listed above in which one or more of residues 22 and 92 are not cysteine residues; SEQ No 21, SEQ No 22, SEQ No 23, SEQ No 24, SEQ No 25, SEQ No 26, SEQ No 27, SEQ No 28 and SEQ No 29 as depicted in FIG. 3; and
(b) in the case of VL: Con, J48, 33, I21R33, I21R33VHI21VL, Con 33 and I21R33(VHC22S;C92S) as depicted in FIG. 3 and designated SEQ 11, SEQ 12, SEQ 13, SEQ 17, SEQ 18, SEQ 19, SEQ 20 respectively.

In a further aspect still the present invention provides an antibody molecule for functionally inactivating activated RAS within an intracellular environment wherein the antibody comprises a heavy chain variable domain and a light chain variable domain, wherein the heavy chain variable domain and the light chain variable domain of the antibody comprise any of the amino acid sequences selected from the group consisting of Con, J48, 33, I21R33, I21R33VHI21VL, Con 33 and I21R33(VHC22S;C92S) as depicted in FIG. 3 and designated SEQ 1, SEQ 2, SEQ 3, SEQ 7, SEQ 8, SEQ 9 and SEQ 10 respectively in the case of variable heavy chain domains; or any of the sequences listed above in which one or more of residues 22 and 92 (according to Kabat numbering) are not cysteine residues; and the corresponding light chain domains as depicted in FIG. 3.

Advantageously, the antibodies according to this aspect of the invention comprise those heavy chain variable domains and the corresponding light chain domains selected from the group consisting of I21R33, I21R33VHI21VL, Con 33 and I21R33 (VHC22S,C92S).

In a preferred embodiment of this aspect of the invention, the antibodies are scFv molecules. In an alternative embodiment of the above aspects of the invention, the antibodies for functionally inactivating mutant RAS are single variable chain domain only antibodies (IDabs). In a most advantageous embodiment of the invention, the IDab is a heavy chain only IDab.

According to the above aspects of the invention, the term ‘functionally inactivating activated RAS’ means that the cell transforming ability of activated RAS is inhibited. By the term ‘inhibited’ it is meant that the cell transforming ability of activated RAS is inhibited as compared with a suitable control in which control cells are not treated with an antibody of the present invention. Advantageously, the cell transforming ability of activated RAS is inhibited by 20% as compared with a suitable control. More advantageously, it is inhibited by 30%, 40%, 50%, 60%, 70%, 80% or 90%. In a most preferred embodiment of this aspect of the invention, the cell transforming ability of activated RAS is inhibited by 100% as compared with a suitable control.

As referred to herein the term the ‘transforming ability’ (of activated RAS) refers to the ability of activated RAS to induce cells to lose their normal growth controls. For example ‘transformed cells’ undergo endless replication and exhibit loss of contact inhibition. That is, the cell divides in an uncontrollable way and can not recognise its own natural boundary. Cells once transformed often form bundles of cells and thus form tumours (tumourigenic transformation). One or more transformed cells may break away from the tumour resulting in further tumour formation (that is the tumour may metastasise).

The term ‘suitable for functionally inactivating activated RAS’ means that an antibody according to the present invention must be of suitable in vivo solubility and antigen binding affinity so that the intracellular antibody is capable of selectively binding activated RAS and consequently the activated RAS is functionally inactivated as herein defined.

The present inventors have surprisingly found that the sequences of the CDRs which determine the specificity of interaction with activated RAS.

Thus, in a further aspect still the present invention provides a single variable domain type anti-activated RAS intracellularly binding antibody comprising a set of variable heavy or light chain domain CDRs selected from the group shown in FIG. 3 and depicted SEQ No: 1a, b and c; SEQ No 2 a, b and c; SEQ No 3, a, b, and c; SEQ No: 11a, b and c; SEQ No 12 a, b and c; and SEQ No 13, a, b, c; SEQ No 21 a, b and c; SEQ No 22 a, b and c; SEQ No 23 a, b and c; SEQ No 24 a, b and c; SEQ No 25 a, b and c; SEQ No 26 a, b and c; SEQ No 27 a, b and c; SEQ No 28 a, b and c; SEQ No 29a, b and c.

Advantageously, according to the above aspect of the invention, a heavy chain variable domain only anti-activated RAS intracellularly binding antibody (IDab) comprises a set of variable heavy chain domain CDRs selected from the group shown in FIG. 3 and depicted SEQ NO 3 a, b and c.

In an alternative embodiment of the above aspect of the invention, the antibody is a single variable heavy chain domain only (IDab) comprising a set of variable heavy chain domain CDRs selected from the group shown in FIG. 3 and depicted SEQ No 21 a, b and c or SEQ No 22 a, b and c.

In an alternative embodiment of the above aspect of the invention, a light chain variable domain only anti-activated RAS intracellularly binding antibody comprises a set of variable heavy chain domain CDRs selected from the group shown in FIG. 3 and depicted SEQ NO 13 a, b and c.

In a further aspect still the present invention provides an anti-activated RAS intracellularly binding antibody comprising at least one light and at least one heavy chain domain wherein the antibody comprises those variable heavy chain domain CDRs selected from the group shown in FIG. 3 and depicted SEQ NO: 1a, b and c; SEQ No 2 a, b and c; and SEQ No 3, a, b, c; and the corresponding light chain domain CDRs selected from the group shown in FIG. 3 and depicted SEQ NO: 1 a, b and c; SEQ No 12 a, b and c; and SEQ No 13, a, b, c respectively.

The present inventors have found that particular CDR sequences depicted in FIG. 3 confer upon an antibody the ability to bind specifically to activated RAS within an intracellular environment.

Thus in a further aspect, the present invention provides those variable domain CDRs selected from those amino acid sequences shown in FIG. 3 and depicted SEQ NO: 1a, b and c; SEQ No 2 a, b and c; SEQ No 3, a, b, c; SEQ 11 a, b, c; SEQ 12 a, b, c, SEQ 13 a, b, c; SEQ No 21 a, b and c; SEQ No 22 a, b and c; SEQ No 23 a, b and c; SEQ No 24 a, b and c; SEQ No 25 a, b and c; SEQ No 26 a, b and c; SEQ No 27 a, b and c; SEQ No 28 a, b and c; SEQ No 29 a, b and c and which when attached to their respective heavy or light chain variable domain framework regions amino acid sequences to generate an intracellularly functional antibody, confer upon the resultant antibody the ability to selectively bind to activated RAS within an intracellular environment.

According to the above aspect of the invention, the intracellularly functional antibody may be a single domain type antibody such as a IDAb. Advantageously the Dab is a heavy chain variable domain IDAb. In an alternative embodiment of the above aspect of the invention the antibody comprises both light and heavy chain variable domains. Advantageously, the antibody is an scFv.

As herein defined the term ‘intracellularly functioning’ (antibody) means that the antibody when expressed within an intracellular environment is both soluble and thermodynamically stable. In addition an ‘intracellularly functioning (antibody) is conformationally similar to that of an antibody within its native environment. That is, it is in a conformation which permits a specific interaction of the antibody via the CDRs with one or more antigens.

According to the above aspect of the invention, the term ‘intracellularly functional antibody’ means that the antibody is of sufficient intracellular stability and solubility so that the CDRs of the variable domains are capable of interacting specifically with their one or more antigens within an intracellular environment.

In a further aspect, the present invention provides a nucleic acid construct encoding any one or more antibody molecules and/or CDR sequences according to the present invention.

In a further aspect still, the invention provides a vector comprising a one or more nucleic acid constructs according to the invention.

In yet a further aspect, the present invention provides a host cell transformed with a vector according to the invention.

The inventors consider that antibodies molecules and/or nucleic acid constructs encoding them will be of significant therapeutic value.

Thus, in a further aspect still, the present invention provides a composition comprising any of those molecules selected from the group consisting of the following: an antibody molecule according to the invention, one or more CDRs of the invention and a nucleic acid construct according to the invention and a pharmaceutically acceptable carrier, diluent or exipient.

The present inventors have found that characteristics of the framework sequences determine the intracellular solubility of an intracellularly expressed anti-activated RAS antibodies generated from those sequences. Preferred framework sequences are shown in FIG. 3 and designated Con, I21, and I21R33.

Thus in a further aspect the present invention provides a method for generating an antibody molecule which is capable of specifically binding to activated RAS and/or functionally inactivating activated RAS within an intracellular environment comprising the step of synthesising the antibody from a variable chain domain comprising any of those amino acids sequences selected from the group shown in FIG. 3 and designated for VH: SEQ No 1, 2, 3, 7, 8, 9, 10 or from any of the listed VH sequences in which one or more of residues 22 and 92 (according to Kabat numbering) are not cysteine residues; and/or synthesising the antibody from a light chain domain variable comprising any of those amino acids selected from the group shown in FIG. 3 and designated SEQ 11, 12, 13, 17, 18, 19 and 20 and depicted Con, J48, 33, I21R33, I21R33VHI21VL, Con33, I21R33 (VHC22S, C92S).

According to the above aspects of the invention, the term ‘synthesising the antibody’ includes within its scope the selection of whole/intact antibodies comprising the sequences referred to above, and/or the selection of antibody fragments comprising the sequences referred to above and their subsequent assembly. Furthermore, the term includes within its scope mutating suitable sequences at the amino acid level or nucleic acid level, in order to generate the sequences referred to above. Mutation may take the form of a substitution, deletion, inversion or insertion. Advantageously the mutation will be a substitution. Methods for performing mutagenesis and manipulation of nucleic acid or amino acid sequences involve standard laboratory techniques and will be familiar to those skilled in the art.

In addition the term ‘synthesising the antibody’ includes within its scope assembling de novo or synthesising de novo a nucleic acid construct encoding the various sequences or fragments thereof, referred to above. The synthesis of nucleic acid may include a PCR based approach. Those skilled in the art will be aware of other suitable methods for the synthesis of nucleic acid encoding the sequences referred to above.

According to the above aspect of the invention, advantageously the method comprises the step of synthesising an scFv from a variable light chain domain selected from the group shown in FIG. 3 and designated SEQ No: 11, 12, 13, 17, 18, 19 and 20 respectively and/or a heavy chain variable domain selected from the group shown in FIG. 3 and depicted SEQ No: 1, 2, 3, 7, 8, 9 and 10.

In a further preferred embodiment of the above aspect of the invention, the method comprises the step of synthesising a IDab from a variable light chain domain selected from the group shown in FIG. 3 and designated SEQ No: 11, 12, 13, 17, 18, 19 and 20 respectively or a heavy chain variable domain selected from the group shown in FIG. 3 and depicted SEQ No: 1, 2, 3, 7, 8, 9 and 10.

In a further aspect still, the present invention provides an antibody obtained using the method of the present invention.

Advantageously, the antibody is a IDab as herein defined or an scFv.

The antibodies according to the present invention are of particular use for in vivo prophylactic and therapeutic purposes. In particular, the present inventors have found that particular antibodies according to the invention are capable of inhibiting the ability of activated RAS to induce transformation in cells.

Thus, in a further aspect still the present invention provides the use of an antibody molecule comprising a light and/or heavy chain variable domain comprising any of those amino acids sequences selected from the group shown in FIG. 3 and designated for VH: SEQ No 1, 2, 3, 7, 8, 9, 10; or from any of those listed VH sequences in which one or more of residues 22 and 92 (according to Kabat numbering) are not cysteine residues or any of those amino acid sequences selected from the group shown in FIG. 3 and designated in the case of VH: SEQ No 21, SEQ No 22, SEQ No 23, SEQ No 24, SEQ No 25, SEQ No 26, SEQ No 27, SEQ No 28 and SEQ No 29; and/or a variable light chain domain comprising any of those amino acids selected from the group shown in FIG. 3 and designated SEQ 11, 12, 13, 17, 18, 19 and 20 and depicted Con, J48, 33, I21R33, I21R33VHI21VL, Con33, I21R33 (VHC22S, C92S) respectively in the preparation of a medicament for specifically binding activated RAS and/or inhibiting the in vivo functional activity of activated RAS within an intracellular environment.

Antibodies suitable for use according to the above aspect of the invention may comprise light and heavy chain variable domains or may be single domain type antibodies, such as Dabs. Preferred antibodies for such use are single domain type antibodies comprising one or more heavy chain variable domains selected from the group comprising of Con, 33, I21R33, I21R33VHI21VL and I21R33 (VHC22S;C92S) and identified as SEQ 1, 7, 8, 10, 21, 22, 23, 24, 25, 26, 27, 28 and SE No 29 as depicted in FIG. 3 respectively and those heavy and light chain antibodies comprising the same heavy chain variable domains referred to above along with their corresponding light chains shown in FIG. 3.

The inventors have shown that the antibodies according to the invention are effective in inhibiting the ability of activated RAS to transform cells. Reports suggest that approximately 30% of all cancers currently known are RAS associated cancers. Thus, the anti-RAS antibodies of the invention show great potential in the prophylaxis and/or treatment of RAS associated cancer.

Thus, in a further aspect still, the present invention provides a method for the treatment of RAS associated cancer in a patient comprising the steps of administering to the patient in need of such treatment a therapeutically effective amount of one or more antibody molecule/s comprising a light and/or heavy chain variable domain comprising any of those amino acids sequences selected from the group shown in FIG. 3 and designated for VH: SEQ No 1, 2, 3, 7, 8, 9, 10; or from any of those listed VH sequences in which one or more of residues 22 and 92 (according to Kabat numbering) are not cysteine residues, any of those amino acids sequences selected from the group shown in FIG. 3 and designated for VH: SEQ No 21, 22, 23, 24, 25, 26, 27, 28 and SEQ No 29; and/or a variable chain domain comprising any of those amino acids selected from the group shown in FIG. 3 and designated SEQ 11, 12, 13, 17, 18, 19 and 20 and depicted Con, J48, 33, 121R33, 21R33VHI21VL, Con33, I21R33 (VHC22S, C92S) respectively.

Antibodies suitable for use according to the method above may comprise light and heavy chain variable domains or may be single domain type antibodies such as Dabs. Preferred antibodies for such use are single domain type antibodies comprising one or more heavy chain variable domains (IDabs) selected from the group comprising of Con, 33, I21R33, I21R33VHI21VL and I21R33 (VHC22S;C92S) and identified as SEQ 1, 7, 8, 10, SEQ No 21, 22, 23, 24, 25, 26, 27, 28 and SEQ No 29; respectively as shown in FIG. 3 and those heavy and light chain antibodies comprising the same heavy chain variable domains referred to above along with their corresponding light chains. Advantageously the antibody is an scFv molecule.

In a further aspect still, the present invention provides the use of an antibody molecule comprising a light and/or heavy chain variable domain comprising any of those amino acids sequences selected from the group shown in FIG. 3 and designated for VH: SEQ No 1, 2, 3, 7, 8, 9, 10 or from any of those listed VH sequences in which one or more of residues 22 and 92 (according to Kabat numbering) are not cysteine residues or any of those amino acid sequences shown in FIG. 3 and depicted SEQ No 21, 22, 23, 24, 25, 26, 27, 28 and SEQ No 29; and/or comprising a variable light chain domain comprising any of those amino acids selected from the group shown in FIG. 3 and designated SEQ 11, 12, 13, 17, 18, 19 and 20 and depicted Con, J48, 33, I21R33, I21R33VHI21VL, Con33, I21R33 (VHC22S, C92S) respectively, in the preparation of a medicament for specifically binding activated RAS and/or inhibiting the in vivo functional activity of activated RAS within an intracellular environment.

In a preferred embodiment of the above aspect of the invention, the antibody is a single domain type antibody. Advantageously, it is a IDab comprising a heavy chain variable domain. Preferably those antibodies comprising at least a heavy chain variable domain comprise any one or those amino acid sequences selected from the group consisting of the following: I21R33 and designated SEQ 7 (VH) and Con 33 designated SEQ 9 (VH) and shown in FIG. 3.

In a further preferred embodiment of the above aspect of the invention, the use according to the above aspect of the invention is of an anti-activated RAS scFv.

### DETAILED DESCRIPTION OF THE INVENTION

Definitions

Immunoglobulins molecules, according to the present invention, refer to any moieties which are capable of binding to a target. In particular, they include members of the immunoglobulin superfamily, a family of polypeptides which comprise the immunoglobulin fold characteristic of antibody molecules, which contains two beta sheets and, usually, a conserved disulphide bond. Members of the immunoglobulin superfamily are involved in many aspects of cellular and non-cellular interactions in vivo, including widespread roles in the immune system (for example, antibodies, T-cell receptor molecules and the like), involvement in cell adhesion (for example the ICAM molecules) and intracellular signalling (for example, receptor molecules, such as the PDGF receptor). The present invention relates to antibodies or scFv molecules. Antibodies as used herein, refers to complete antibodies or antibody fragments capable of binding to a selected target, and including Fv, ScFv, Fab′ and F(ab′)₂, monoclonal and polyclonal antibodies, engineered antibodies including chimeric, CDR-grafted and humanised antibodies, and artificially selected antibodies produced using phage display or alternative techniques. Small fragments, such as Fv and ScFv, possess advantageous properties for diagnostic and therapeutic applications on account of their small size and consequent superior tissue distribution. Preferably, the antibody is a single domain antibody (IDab) or scFv. As herein defined the term ‘antibody’ includes within its scope molecules which comprise an antigen binding moiety comprising at least one heavy chain variable domain and at least one antibody constant region domain.

As herein defined a ‘Dab/IDab’ is a single variable heavy chain domain or a single variable light chain domain optionally attached to a ‘bulking group’. The ‘bulking group’ as herein defined may comprise one or more antibody constant region domains. Alternatively, the ‘bulking group’ may comprise components of non-immunoglobulin origin. These may include cytotoxins, fluorescent or other forms of labels. Those skilled in the art will appreciate that this list is not intended to be exhaustive. Most advantageously, a ‘Dab’ according to the present invention comprises a single heavy chain variable domain attached to one or more constant region domains as herein defined. For the avoidance of any doubt, a Dab may comprise a light or heavy chain variable domain alone. In a preferred embodiment of the invention, an IDab as described herein comprises a heavy chain variable domain only.

Heavy chain variable domain refers to that part of the heavy chain of an immunoglobulin molecule which forms part of the antigen binding site of that molecule. The VHIII subgroup describes a particular sub-group of heavy chain variable regions (the VHIII). Generally immunoglobulin molecules having a variable chain amino acid sequence falling within this group possess a VH amino acid sequence which can be described by the VHIII consensus sequence in the Kabat database.

Light-chain variable domain refers to that part of the light chain of an immunoglobulin molecule which forms part of the antigen binding site of that molecule. The VkI subgroup of immunoglobulin molecules describes a particular sub-group of variable light chains. Generally immunoglobulin molecules having a variable chain amino acid sequence falling within this group possess a VH amino acid sequence which can be described by the V_{K}I consensus sequence in the Kabat database.

Framework region of an immunoglobulin heavy and light chain variable domain. The variable domain of an immunoglobulin molecule has a particular 3 dimensional conformation characterised by the presence of an immunolgobulin fold. Certain amino acid residues present in the variable domain are responsible for maintaining this characteristic immunoglobulin domain core structure. These residues are known as framework residues and tend to be highly conserved.

CDR (complementarity determining region) of an immunoglobulin molecule heavy and light chain variable domain describes those amino acid residues which are not framework region residues and which are contained within the hypervariable loops of the variable regions. These hypervariable loops are directly involved with the interaction of the immunoglobulin with the ligand. Residues within these loops tend to show less degree of conservation than those in the framework region.

Intracellular means inside a cell, and the present invention is directed to those immunoglobulins which will bind to ligands/targets selectively within a cell. The cell may be any cell, prokaryotic or eukaryotic, and is preferably selected from the group consisting of a bacterial cell, a yeast cell and a higher eukaryote cell. Most preferred are yeast cells and mammalian cells. As used herein, therefore, “intracellular” immunoglobulins and targets or ligands are immunoglobulins and targets/ligands which are present within a cell (including the cytoplasm and the nucleus). In addition the term ‘Intracellular’ refers to environments which resemble or mimic an intracellular environment. Thus, “intracellular” may refer to an environment which is not within the cell, but is in vitro. For example, the method of the invention may be performed in an in vitro transcription and/or translation system, which may be obtained commercially, or derived from natural systems.

Consensus sequence of V_{H} and V_{L} chains in the context of the present invention refers to the consensus sequences of those V_{H} and V_{L} chains from immunoglobulin molecules which can bind selectively to a ligand in an intracellular environment. The residue which is most common in any one given position, when the sequences of those immunoglobulins which can bind intracellularly are compared is chosen as the comparing the residues for all the intracellularly binding immunoglobulins, at each position in turn, and then collating the data. In this case the sequences of 18 immunoglobulins was compared.

Specific (antibody) binding in the context of the present invention, means that the interaction between the antibody and the ligand are selective, that is, in the event that a number of molecules are presented to the antibody, the latter will only bind to one or a few of those molecules presented. Advantageously, the antibody-ligand interaction will be of high affinity. The interaction between immunoglobulin and ligand will be mediated by non-covalent interactions such as hydrogen bonding and Van der Waals forces.

A repertoire in the context of the present invention refers to a set of molecules generated by random, semi-random or directed variation of one or more template molecules, at the nucleic acid level, in order to provide a multiplicity of binding specificities. In this case the template molecule is one or more of the VH and/or VL domain sequences herein described. Methods for generating repertoires are well characterised in the art.

The term ‘activated RAS’ refers to the form of RAS which is capable of inducing transformation of a cell. Thus, according to the present invention, the term ‘activated RAS’ is synonymous with the ‘oncogenic form of RAS’. As referred to herein the term ‘transforming ability’ (of activated RAS) means the ability of activated RAS to induce cells to lose their normal growth controls. For example ‘transformed cells undergo endless replication and exhibit loss of contact inhibition. That is, the cell divides in an uncontrollable way and can not recognise its own natural boundary. Cells once transformed often form bundles of transformed cells and thus form tumours (tumourigenic transformation). One or more transformed cells may break away from the tumour resulting in further tumour formation (that is the tumour may metastasise).

The term ‘specific binding to activated RAS’ means that within a mixture of reagents comprising activated RAS in addition to other alternative antigens, only activated RAS is bound. Thus the binding of an anti-RAS antibody according to the present invention is selective for activated RAS. The specific binding of an antibody according to the present invention to activated RAS may be of high affinity or low affinity. For example, the specific interaction of scFv I21 with activated RAS is of low affinity whereas the specific interaction of scFv 33 with activated RAS is of high affinity.

The term ‘intracellularly functional antibody’ means that the antibody is of sufficient intracellular stability and solubility so that the CDRs of the variable domains are capable of interacting specifically with their one or more antigens within an intracellular environment.

The term ‘suitable for functionally inactivating activated RAS’ means that an antibody according to the present invention must be of suitable in vivo solubility, stability and antigen binding affinity so that the intracellular antibody is capable of selectively binding activated RAS and consequently the activated RAS is functionally inactivated as herein defined. The term ‘functionally inactivating activated RAS’ means that the cell transforming ability of activated RAS is inhibited. By the term ‘inhibited’ it is meant that the cell transforming ability of activated RAS is inhibited as compared with a suitable control in which control cells are not treated with an antibody of the present. Advantageously, the cell transforming ability of activated RAS is inhibited by 20% as compared with a suitable control. More advantageously, it is inhibited by 30%, 40%, 50%, 60%, 70%, 80% or 90. In a most preferred embodiment of this aspect of the invention, the cell transforming ability of activated RAS is inhibited by 100% as compared with a suitable control.

### BRIEF DESCRIPTION OF THE FIGURES AND TABLES

Table 1. IDab Library Screening Data.

Two different IDab-VP16 libraries were screened with two antigen baits (HRASG12V and ATF-2) as LexA-DBD fusions. Library 1 had randomised VH CDR 2 and 3, while library 2 had randomised VH CDR1, 2 and 3. The primary screening results are shown as the initial number of clones screened in yeast L40 with the antigen bait and the numbers of colonies growing on histidine-deficient plates (HIS-growth) and the corresponding proportion causing β-gal activation (β-gal positive).

Table 2. Affinity Measurements of anti-RAS IDab Proteins Using a BIAcore.

His-tagged antibody fragments were produced by expression in bacteria and purified by Ni-NTA agarose affinity chromatography. Biosensor measurements were made using a BIAcore 2000. The table summarises the values of association (Kon) and dissociation rates (Koff) together with calculated equilibrium dissociation constants (Kd) using BIA-evaluation 2.1 software. At high IDab concentrations, non-specific interactions between IDab and antigen were detected.

scFv33 (Tanaka & Rabbitts, 2003); scFvI21R33VHI21VL is an scFv derivative of scFv33 with VH framework regions of scFvI21, VH CDR1, 2 and 3 of scFv33 and VL of I21 (Tanaka & Rabbitts, 2003); IDabs #3, #10 and #12 are intrabodies isolated from the IDab libraries using HRASG12V as a bait.

FIG. 1. Intracellular antibody capture of anti-RAS scFv.

2.7×10¹³ clones from three different phage libraries (de Wildt et al., 2000; Sheets et al., 1998) (total diversity 7.0×10⁹) were screened with purified HaRASG12V antigen in vitro. 1.18×10⁶ phage were recovered, phagemid DNA was prepared and scFv fragments cloned into the yeast vector pVP16 to make sub-library of 4.13×10⁶ clones. 8.45×10⁷ yeast clones were screened in yeast L40 strain expressing the LexA-RASG12V bait. 428 colonies grew on histidine selective plates and showed strong activation of the lacZ gene, determined by β-gal filter assay. All prey plasmids were isolated from histidine-independent and β-gal positive yeast colonies and were fingerprinted by digestion with restriction enzymes, BstN1, Msp1, Mbo1, RsaI or Hinf1 to identify the differing scFv clones. Subsequently 57 scFv clones which had different DNA fingerprinting patterns were re-tested in yeast with LexA-RASG12V bait and three scFv (which were originated from different libraries were isolated). Of these three anti-RAS scFv, only two detectably bound RAS protein in a mammalian reporter assay.

FIG. 2. Interaction of anti-RAS scFv with RAS protein in mammalian cells.

A. Luciferase Assay; COS7 cells were transiently co-transfected with various scFv-VP16 activation domain fusions and the GAL4-DBD bait plasmid pM1-HRASG12V (closed boxes) or pM1-lacZ (open boxes), together with the firefly luciferase reporter plasmid pG5-Luc and an internal Renilla luciferase control plasmid pRL-CMV. scFv-VP16 prey vectors were used expressing anti-RAS scFv33, J48 and I21 or anti-β-gal scFvR4 (Martineau et al., 1998). The luciferase activities were measured 48 hours after transfection using Dual Luciferase Assay System (Promega) and a luminometer. The luciferase activities of each assay were normalised to the Renilla luciferase activity (used as internal control for the transfection efficiency). The fold luciferase induction level is shown with the activity of each scFv-VP16 with non-relevant bait taken as baseline.

B. In situ immunofluorescence study; COS7 cells were transiently co-transfected with pEF-myc-nuc-scFv J48 (anti-RAS scFv) or scFvR4 (anti-β-gal scFv) and pHM6-RAS vectors expressing the RAS antigen. After 48 hours, cells were fixed and stained with 9E10 monoclonal antibody (detecting the myc tagged scFv) and rabbit anti-HA tag polyclonal serum, followed by secondary fluorescein conjugated anti-mouse and Cy3 conjugated anti-rabbit antibodies, respectively. The staining patterns were examined using a BioRadiance confocal microscope. Co-location of antigen and ICAb fluorescence was found for scFv J48 co-expressed with RAS.

Green (fluorescein)=fluorescence of scFv; Red (Cy3)=fluorescence of antigen

FIG. 3. Sequence of anti-RAS intracellular antibodies.

3(A) The Sequences of Anti-RAS scFv

(A)The nucleotide sequences were obtained and the derived protein translations (shown as single letter code) were aligned. Dashes in framework (FR) represent identities with the consensus (CON) sequence (derived from anti-BCR and anti-ABL scFv isolated by the IAC method (Tse et al., 2002)). The numbers indicate the reference positions of the residues, according to the system by Lefranc et al (Lefranc and Lefranc, 2001) (top column number, indicated as IMGT) and Kabat et al (Kabat et al., 1991) (second column, Kabat). The 15 residues of the linker, (GGGGS)₃ between the heavy chain of variable domain (VH) and light chain (VL) are not shown. The complementarily determining regions (CDR) are highlighted on grey background and demarcated from framework regions (FR). Three anti-RAS intracellular scFv are designed as 33, J48 and I21. All anti-RAS scFv belong to the VH3 subgroup of heavy chain and Vκ1 subgroup of light chain shown in the middle (designed VH3 or VκI) from the Kabat database (Kabat et al., 1991) or IGVH3 and IGVK1 from the Lefranc database(Lefranc and Lefranc, 2001). The mutated anti-RAS scFv are shown designed as I21K33, I21R33, I21R33VHI21VL, con33, and I21R33VH (C22SC92S). I21K33 comprises the CDRs of scFv33 in the I21 framework and I21R33 is identical except for a mutation Lys94Arg; I21R33VH21VL comprises the VH domain of 21R33 fused to the VL domain of I21; con33 has all six CDRs of scFv33 in the canonical consensus framework (Tse et al., 2002); I21R33VH (C22S;C92S) is a mutant of clone I21R33 with the mutations CYS22SER and CYS92SER of the VH domain. There are only four amino acid differences (at positions H1, H5, L0, and L3) between consensus and 121R framework regions. A, b and c represent CDR sequences.

FIG. 3(B). VH CDR protein sequences of IDabs isolated from intrabody library screening

Alignment of derived protein sequences of complementarity determining regions (CDR) of selected IDab intrabody clones obtained by screening the single domain libraries with two protein baits viz. HRASG12V and ATF-2 proteins.

(I). The nucleotide sequences of the IDab clones were obtained and the derived protein translations (shown in the single-letter code) were aligned. The IDab CDRs are aligned and compared with those of IDab33 (the highlighted CDR regions of the VH domain are defined by IMGT (the International ImMunoGeneTics, information system at http://imgt.cines.fr) (Lefranc & Lefranc, 2001) (grey highlighted in IDab33, top line) and by Kabat et al. (underlined in IDab33, top line) (Kabat et al., 1991)). In the sequences of the IDabs selected from the libraries, only those regions which were randomised by the PCR mutagenesis are highlighted with grey. Note that the anti-RAS IDabs clones 11 to 19 originated from IDab library 2 and these have all three CDRs mutated and hence the highlighted region of CDR1 as well as CDR2 and 3 in the sequences derived from those clones. SEQ No 21 is clone 3; SEQ No 22 is clone 6, SE No 23 is clone number 7; SEQ No 24 is clone number 10; SEQ No 25 is clone 12; SEQ No 26 is clone 13; SEQ No 27 is clone 17; SEQ No 28 is clone 18; SEQ No 29 is clone 19. The areas designated a, b and c are the CDR sequences.

(II). The middle panel shows which VH framework each selected IDab originates from. CON=framework from the scFv625 which carries the canonical IAC consensus (Tse et al., 2002). I21R=framework from the scFvI21R33 which has a sequence very close to the canonical consensus (Tanaka & Rabbitts, 2003).

C. Each selected IDab was re-tested in the yeast assay with either the starting bait or the heterologous bait using both histidine dependence (HIS) or β-gal activation assays (β-gal) and scored positive (+) or negative (−) in those assays.

FIG. 4. Periplasmic expression and purification of anti-RAS scFv.

The scFv with pelB leader sequence at N-terminal and His6-tag and myc-tag at C-terminal were expressed periplasmically from the pHEN2-scFv vector in E. coli HB2151 using 1 mM IPTG for 2 hour at 30° C. in 1 litre of 2×TY medium including 100 μg/ml ampicillin and 0.1% glucose. After induction, the cells were harvested and extracted in 4 ml of ice cold 1×TES buffer (0.2 M Tris-HCl (pH 7.5), 0.5 mM EDTA, 0.5 M sucrose) and a further 6 ml of 1:5 TES buffer was added. The supernatants of cell extracts were used as the soluble periplasmic fraction. The his-tagged scFv were purified by immobilised Ni²⁺ ion chromatography and fractionated by 15% SDS-PAGE and proteins revealed by Coomassie blue staining. The approximate yields of purified anti-RAS scFv33 and J48 were less than 100 μg per 1 litre culture; scFvI21R33, I21R-33VHI21L and I21 more than 3 mg per litre; con33, 1 mg per litre.

E=Complete periplasmic extracts and P=purified scFv; M=Mw markers

FIG. 5. Specific antigen binding and competition ELISA of anti-RAS scFv. Purified HRASG12V-GppNp (4 μg/ml, approximately 200 nM; black boxes) or bovine serum albumin (BSA, 30 mg/ml, approximately 450 μM; grey boxes) were coated on to ELISA plates for 1.5 hours at room temperature. For both sets of wells, 3% BSA in PBS was added for blocking and subsequently purified scFv (450 ng per well) was added and incubated overnight at 4° C. After washing with PBS-0.1% Tween 20, bound scFv was detected with HRP-conjugated anti-poly-histidine antibody (HIS-1, Sigma) and signals quantitated using Emax microplate reader (Molecular Devices). For competition assays (indicated in figure as +), scFv were pre-incubated with HRASG12V-GppNp (8 μg/ml; approx. 400 nM) for 30 min at room temperature before addition to ELISA well.

FIG. 6. Affinity measurements of anti-RAS scFv using BIAcore.

Biosensor measuremenst were made using the BIAcore 2000. Purified scFv from bacterial cultures were used.

A. Sensograms showing the binding of anti-RAS scFv with HRASG12V-GppNp antigen (immobilised 1500 RU). An injection volumes of 40 μl and flow rates of 20 μl/min were used. The purified scFv (10-2000 nM) were loaded on 2 channels of the chip, containing either immobilised HRASG12V-GppNp or no antigen. The sensograms of each measurement were normalised by the resonance of the channel without antigen.

B. The table summarises the value of association rate (Kon) and the dissociation rate (Koff) and calculated equilibrium dissociation constants (Kd) by BIAevaluation 2.1 software.

FIG. 7. Influence of framework residues on the solubility of expressed scFv in COS7 cells.

COS7 cells were transiently transfected with pEF-myc-cyto-scFv expression clones as indicated. Soluble and insoluble proteins were extracted, as described in materials and methods, and fractionated on 15% SDS-PAGE. After electrophoresis, protein were transferred to membranes and incubated with the anti-myc tag monoclonal antibody, 9E10. The migration molecular weight markers (in kDa) are shown on the left. Arrows on the right indicate to the scFv fragment band.

FIG. 8. Improvement of intracellular interaction between anti-RAS ICAbs and RAS antigen by the mutation of framework sequences.

Mammalian two-hybrid antibody-antigen interaction assays were performed in COS7 cells.

A. COS7 were transfected with the pEFBOSVP16-scFv vectors and the pM1-RASG12V, together with the luciferase reporter clones and luciferase levels were determined as described in methods. The upper panel represents normalised fold induction of luciferase signals (zero being taken as signal from prey plasmid without scFv) for scFv-VP16 binding RAS antigen bait. The lower panel shows a Western blot of COS7 cell extracts after the expression of scFv-VP16 fusion proteins. ScFv-VP16 fusion proteins were detected by Western-blot using anti-VP16 (Santa Cruz Biotechnology, 14-5) monoclonal antibody and horseradish peroxidase (HRP)-conjugated anti-mouse IgG antibody.

ICAb scFv used as a control was anti-β-gal R4 (Martineau et al., 1998). scFv33 mutants were (using Kabat et al (Kabat et al., 1991) and number in parenthesis also indicate numbering by Lefranc et al (Lefranc and Lefranc, 2001)) (see FIG. 3)
VH(A74S+S77I): substitutions Ala74(83)Ser and Ser77(86)Thr of VH
VH(D84A): substitution Asp84(96)Ala of VH
VH(R94K): substitution Arg94(106)Lys of VH
VL(0T+V3Q): addition Thr between linker and VL domain plus substitution
Val3(3)Gln of VL
VL(F10S): substitution Phe10(10)Ser of VL
VL(184T): substitution Ile84(100)Thr of VL
VH(Q1E+V5L+A7S+S28T)+VL(G100Q+V104L): substitutions Gln1(1)Glu, Val5(5)Leu, Ala7(7)Ser, Ser28(29)Thr of VH plus Gly100Gln and Val104Leu of VL.

B. COS7 cell two-hybrid antibody-antigen interaction assay using scFv with framework mutations to convert to consensus sequence scaffolds. The various scFv-VP16 prey constructs shown were transiently transfected with GAL4-RASG12V bait plasmid in COS7 cells and the luciferase activities were measured 48 hour after transfection. The fold luciferase activity level are shown in histogram with the activity of no scFv (prey plasmid without scFv) as baseline. The expression levels of scFv-VP16 in soluble fraction of COS7 cells are shown in lower panel. The bands were visualised by Western-blot using anti-VP16 (14-5) antibody and HRP-conjugated anti mouse IgG antibody.

FIG. 9. Inhibition of RAS-dependent NIH3T3 cells transformation activity by anti-RAS scFv.

Mutant HRAS G12V cDNA were subcloned into the mammalian expression vector pZIPneoSV(X) and anti-RAS scFv into pEF-FLAG-Memb vector which has plasma membrane targeting signal at C-terminal of scFv and FLAG-tag at N-terminal to scFv. 100 ng of pZIPneoSV(X)-RASG12V and 2 μg of pEF-FLAG-Memb-scFv were co-transfected into NIH 3T3 cells cloneD4. Two days later, the cells were transferred to 10 cm plates and grown for 14 days in DME medium containing 5% donor calf serum and penicillin and streptomycin. Finally, the plates were stained with crystal violet and foci of transformed cells were counted.

(A) Representative photograph of stained plates. Empty vector in left-top panel indicates co-transfection of pZIPneoSV(X) without RASG12V and pEF-FLAG-Memb without scFv as negative control. No foci formation was observed. The right-top panel indicates pZIPneoSV(X) with RASG12V pEF-FLAG-Memb without scFv as positive control. In the other plates, the RASG12V vector was co-transfected with either pEF-memb-scFvI21 or pEF-memb-scFvI21R33.

(B) Relative percentage of transformation foci was determined as a number of foci normalised to the focus formation induced by pZIPneoSV(X)/HRASG12V and pEF-memb empty vector, which was set at 100. Results shown represent one experiment with each transfection performed in duplicate. Two additional experiments yielded similar results.

FIG. 10. Inhibition of mutant RAS-mediated oncogenic transformation of NIH3T3 cells by anti-RAS IDabs.

Mutant HRASG12V cDNA was cloned into the mammalian expression vector pZIPneoSV(X) and anti-RAS scFvs or IDabs were cloned into the pEF-FLAG-Memb vector (this encodes a protein with a plasma membrane targeting signal fused at the C-terminus of each scFv or IDab and a FLAG-tag fused at the N-terminus). 100 ng of pZIPneoSV(X)-HRASG12V and 2 μg of pEF-FLAG-Memb-scFv or pEF-FLAG-Memb-IDab were co-transfected into low passage NIH3T3 D4 cells using LipofectAMINE™ (Invitrogen). Two days later, the cells were transferred to 10 cm plates. After reaching confluence, cells were maintained for 14 days in DME medium containing 5% donor calf serum and the plates were stained with crystal violet to allow foci of transformed cells to be quantitated.

A. Photographs of representative NIH3T3 growth plates showing transformed foci. Empty vector in the top left panel indicates co-transfection with the pZIPneoSV(X) vector without cloned RAS together with the pEF-FLAG-memb vector without cloned scFv or IDab. The other plates show cultures after transfections of cells with pZIPneoSV(X)-HRASG12V plus the indicated scFv or IDab pEF-FLAG-memb expression vector.

B. A histogram showing relative percentage of transformed foci, estimated as the number of foci normalised to the focus formation induced by pZIPneoSV(X)-HRASG12V together with the pEF-FLAG-Memb empty vector only (the value set at 100%). Results shown represent one experiment, in which each transfection was performed in duplicate (two additional experiments yield similar results).

General Techniques

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4^{th} Ed, John Wiley & Sons, Inc. which are incorporated herein by reference) and chemical methods. In addition Harlow & Lane., A Laboratory Manual Cold Spring Harbor, N.Y., is referred to for standard Immunological Techniques.

De Novo Synthesis of Antibodies

The present inventors have identified variable heavy chain and variable light chain framework residues which determine the in vivo stability and solubility of antibodies comprising them.

Thus the invention provides a provides a method for generating an antibody comprising either one or more heavy chains only or both heavy and light chains which is suitable for intracellular use comprising the step of synthesising the antibody using any of the framework sequences, (or the nucleic acid encoding them) selected from the group consisting of the following: the heavy chain framework region depicted in FIG. 3 as I21 and designated SEQ 4, the heavy chain framework region of the consensus sequence designated SEQ 7 and depicted as I21R33, and the heavy chain framework region of the consensus sequence designated SEQ 1 and depicted as Con; or any of these framework sequences wherein the amino acid residues at positions 22 and 92 according to Kabat are not cysteine residues and additionally synthesising the antibody from the corresponding light chain framework regions as depicted in FIG. 3.

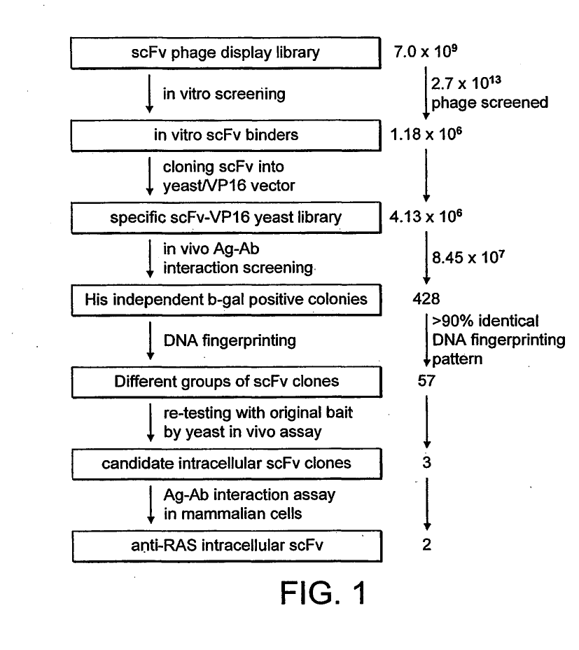

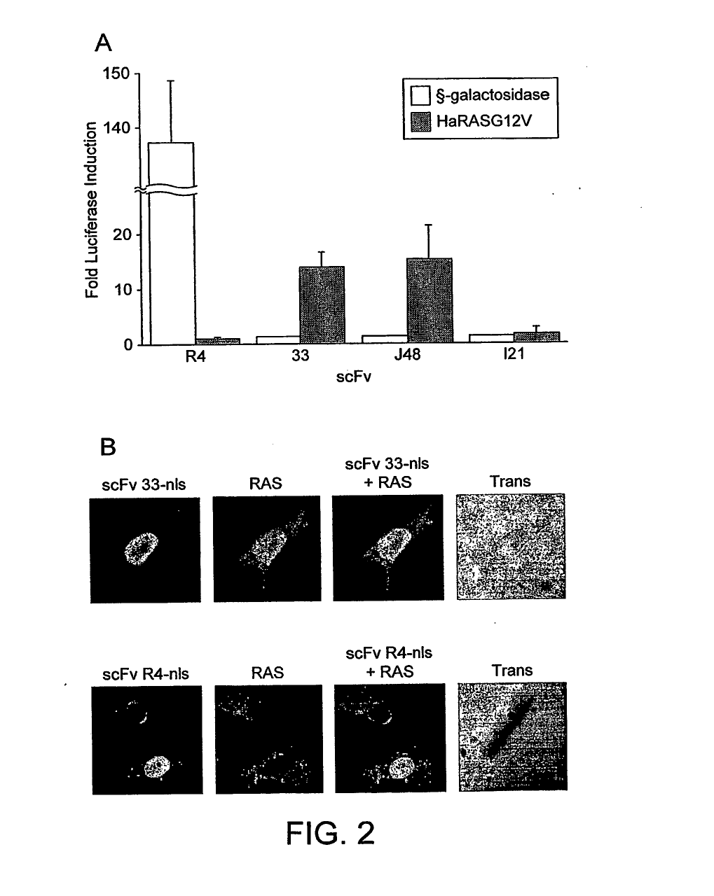

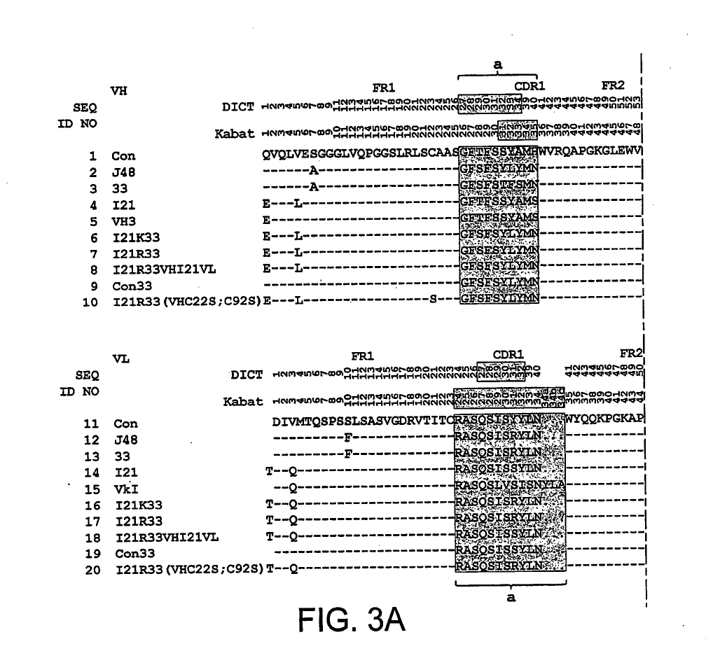

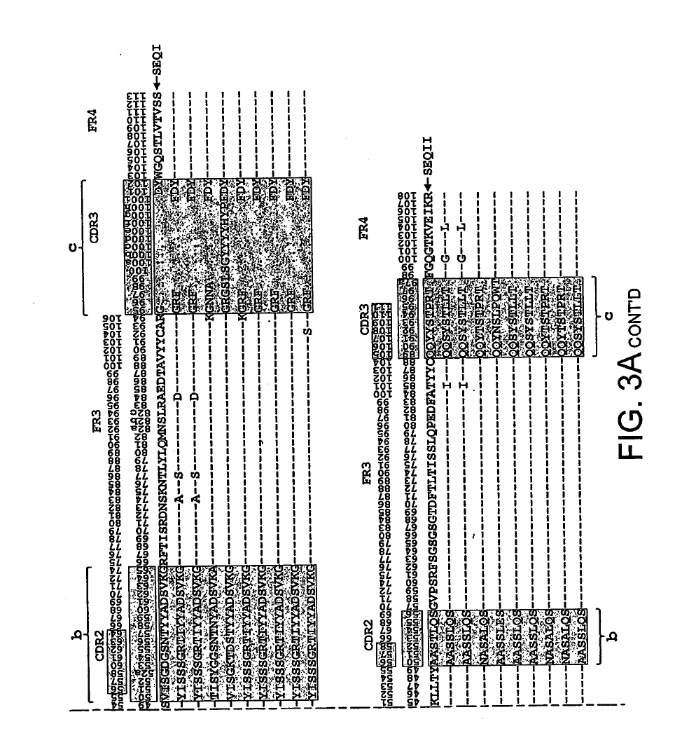

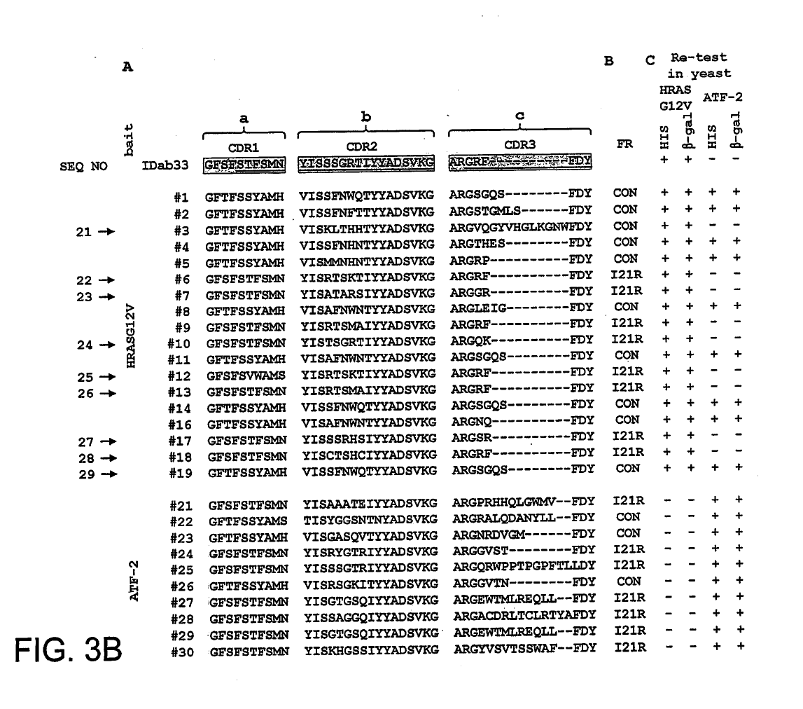

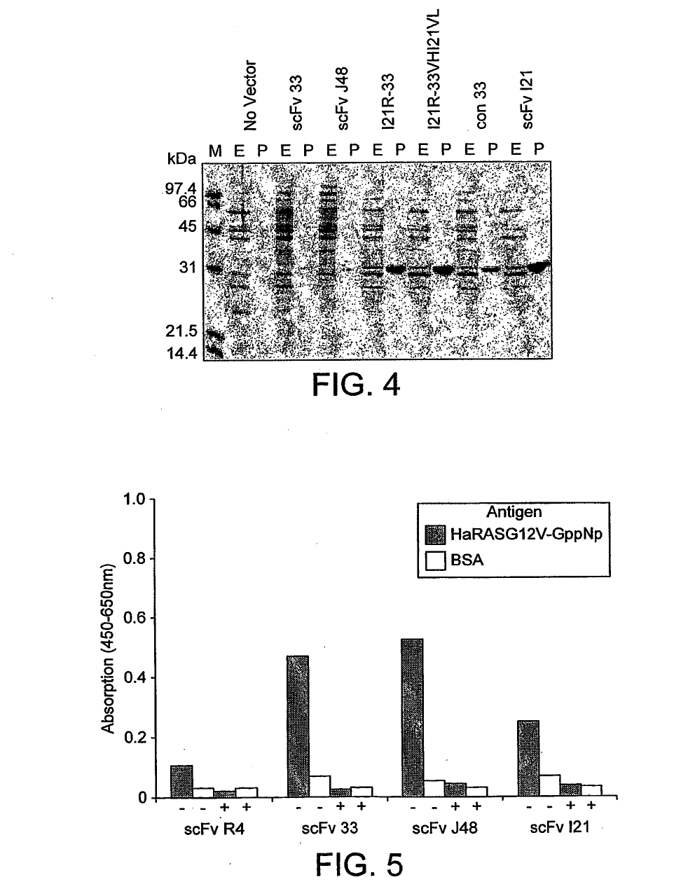

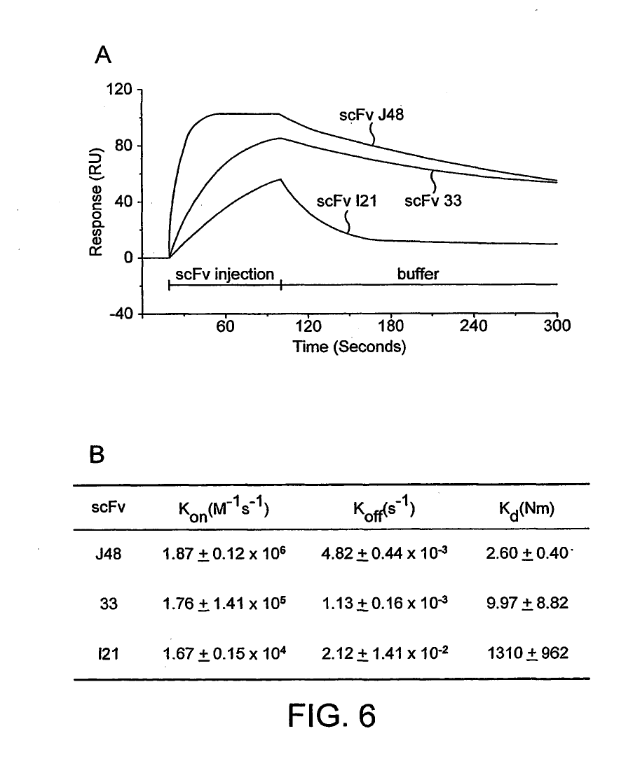

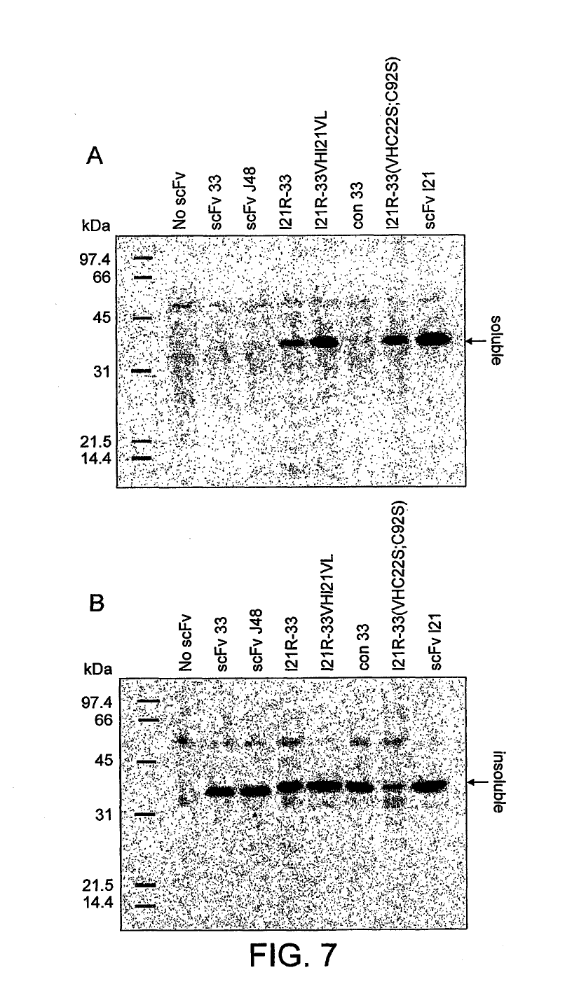

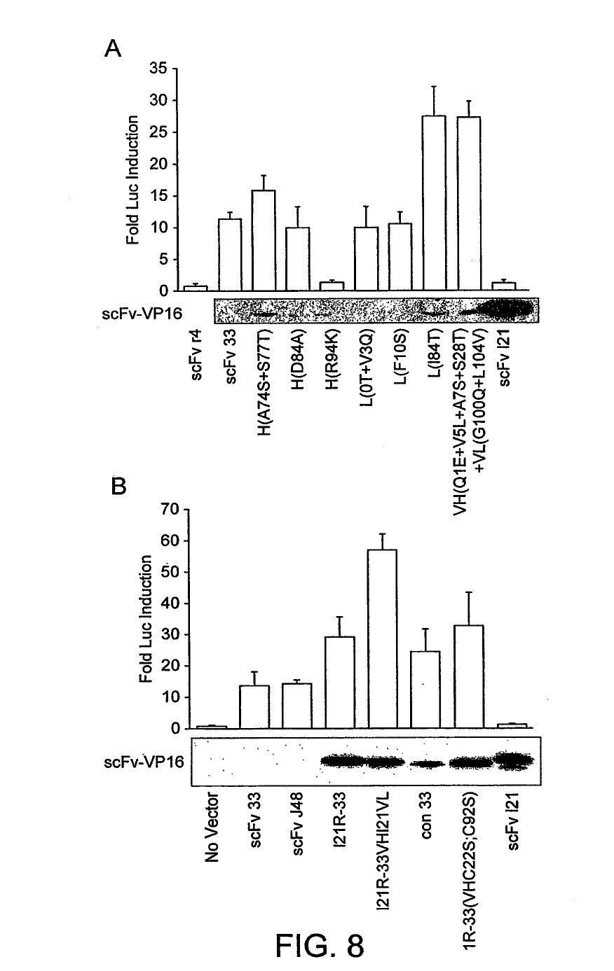

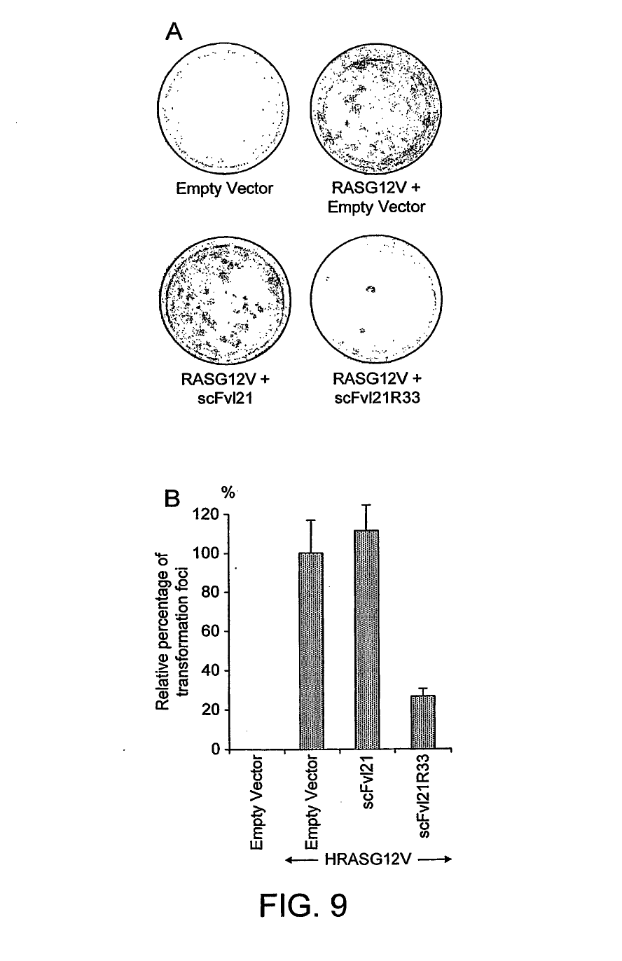

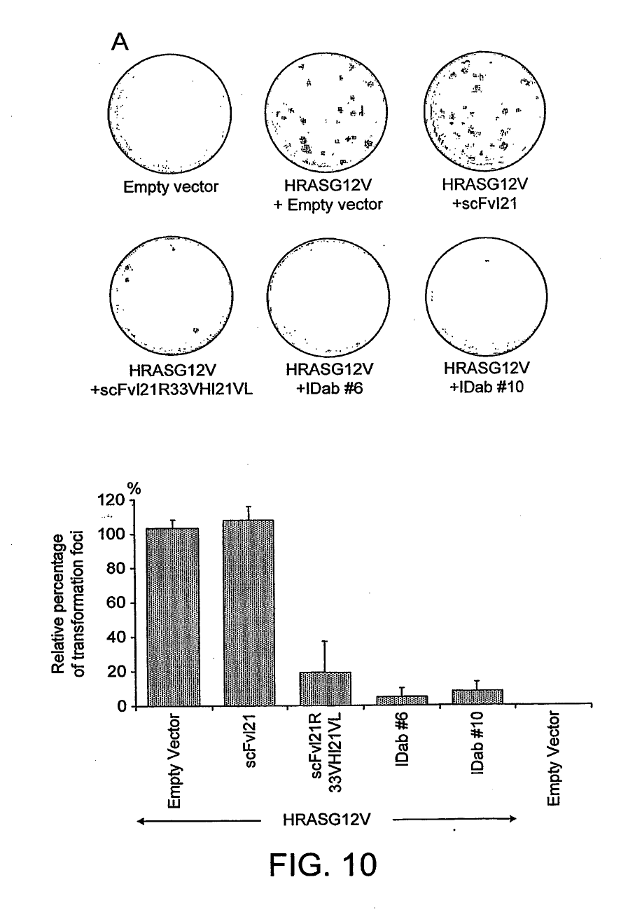

The present inventors have shown that the framework regions of depicted as I21 in FIG. 1 and designated SEQ 4 and 14 and the framework regions of the consensus designated SEQ 1 and 11 in FIG. 1, and SEQ 7 and 17 in FIG. 3 confer upon the an antibody comprising them the conformational stability and solubility required to function within an intracellular environment

In addition the above listed sequences which do not comprise cysteine residues at one or more of positions 22 and 92 according to Kabat (for VH) may also be used to confer upon the an antibody comprising them the conformational stability and solubility required to function within an intracellular environment. In particular those amino acid sequences wherein the cysteine at one or both positions is replaced by serine may be used to confer upon the an antibody comprising them the conformational stability and solubility required to function within an intracellular environment.

Additionally, experiments have shown that those CDRs present on scFv molecules J48 and 33, and designated SEQ 2 and 12 (J48) and SEQ 3 and 13 (33) in FIG. 1 respectively confer upon an intracellularly stable antibody comprising them the ability to bind activated RAS specifically.

Thus, any combination of the framework sequences above with those CDR sequences listed above when combined can be used to generate de novo a Dab or a light and heavy chain antibody which can specifically bind to activated RAS within an intracellular environment.

A suitable method for the de novo synthesis of antibodies is described in our co-pending british application entitled ‘Method for generating Immunoglobulin genes’ which is filed on even date herewith.

Thus, it is reasonable to expect that ICAb libraries can be constructed which are based on the consensus ICAb scaffold and of sufficient diversity to allow primary screening directly in yeast cell assays, without recourse to a preliminary in vitro phage antibody screen with protein antigen. This would provide very clear technical advantages for example that antigens would not need to be expressed in vitro to provide for protein and the requirement for the yeast bait expression for IAC selection is merely DNA sequence. This opens the possibility of using IAC technology to select ICAbs which bind to any protein predicted from genome sequence analysis of any organism e.g. from the human genome sequencing programme.

A key question is the degree of library diversity required to contain effective intracellular antibodies and thus whether sufficient antibody diversity can be made and screened in yeast. The success of the current IAC requires to start with in vitro phage Ab screening of highly diverse libraries. However, such phage antibodies do not necessarily work as intracellular antibody without modification (because of solubility and folding problems in reducing environment) meaning that the effective diversity of these phage scFv libraries, as intracellular antibody libraries, is likely to be less than expected. The construction of specially designed human intracellular antibody libraries using randomised CDRs on the fixed consensus framework should increase effective ICAb diversity. This will make it possible to screen the library for ICAbs without the need for preliminary phage panning step, while keeping full effective diversity and accelerating the screening for intracellular antibodies.

Activated RAS Specific Intracellularly Functioning Antibodies According to the Invention

The present inventors have isolated a series of activated RAS specific antibodies which are capable of specifically binding to activated RAS within an intracellular environment.

(I) The LC Method for the Isolation of Antibodies

The IAC approach has several advantages compared with other screening methods. It is based on the yeast two-hybrid in vivo assay, which works as direct cytoplasmic selection of scFv. In addition, it theoretically allows the selection of antibody fragments (in the experiments described here scFv) against any cytoplasmically expressed antigen, including post-transcriptionally-modified proteins or especially protein complexes, as it allows targeting of antigen in its native form. A further consideration is that the screening process involves verification of candidate intracellular scFv in mammalian cells. By adopting these different bait and reporter systems, false-positive scFv are eliminated. In addition, the mammalian antigen-antibody interaction assay is performed at 37° C., compared with 30° C. in yeast or at room temperature (or at 4° C.) for in vitro phage screen. This step from yeast to mammalian cells makes it possible to select more thermal-tolerant intracellular scFv and because the isolation involves a mammalian assay, higher affinity interactions may be selected which are suitable for competitive binding of the target antigen with endogenous dimerisation molecules.

In this work, the present inventors have applied IAC technology against the activated protein RAS and have isolated specific anti-RAS scFv which bind to this antigen in the cell cytoplasm. Sequence analysis demonstrates that all anti-RAS scFv belong to VH3 and Vκ1 subgroup defined from Kabat database (Kabat et al., 1991) or IGHV3 and IGK1 subgroup defined from IMGT database (Lefranc and Lefranc, 2001). Most ICAb scFv selected which bind the antigens BCR or ABL (Tse et al., 2002) and TAU (Visintin et al., 2002), also belong to same subgroup, supporting the notion these subgroups of VH and Vκ framework can function intracellularly. This observation allowed a consensus framework scaffold to be defined (Tse et al., 2002).

The most important requirements for intracellular antibodies as therapeutic or bioscience research tools is that these antibodies (or antibody fragments) exhibit high stability, good expression levels and are functional within any compartment of mammalian cells. These are severe limitations and few scFv fragments derived from hybridomas are stable under a reducing environment without modification, even if they have good affinity in vitro. The intracellular antibody capture (IAC) technology described here, and previously (Tse et al., 2002; Visintin et al., 2002) overcome these difficulties being based on an in vivo genetic screen for the direct isolation of functional scFv.

(II) Modified IAC Approach (IAC²).

In a modification of the above approach the present inventors used a direct intracellular single domain (IDab) format, based on a previously characterised intrabody consensus scaffold, to generate diverse intrabody libraries for direct in vivo screening. Anti-RAS IDabs isolated this way were found to possess an antigen binding affinity of between 20 nm and 200 nm. Anti-RAS IDabs isolated using this method are described in section (iii) below:
(i) Anti activated RAS antibodies comprising both heavy and light chain variable domains which are capable of specifically binding to activated RAS within an intracellular environment.

The present inventors have isolated antibodies comprising both heavy and light chains which bind specifically to activated RAS within an intracellular environment. The inventors found that some of these antibodies bind with low affinity and others with high affinity for antigen. In addition, the inventors found that some of these antibodies have high solubility levels within an intracellular environment and others have low solubilitys.

For example, using IAC screening for anti-RAS scFv, one scFv (121) shows high yields in bacteria periplasm, high solubility in mammalian cells but poor affinity of interaction in mammalian cells whilst other scFv (scFv33 and J48) have high affinity but relatively low yields of soluble expressed protein. The I21 scFv framework sequence however conforms closely with the consensus framework (Tse et al., 2002) shown in FIG. 3, in both the VH and VL regions. Therefore, these results indicate that the consensus framework identified in FIG. 1 as SEQ 1 and 11 and also the I21 framework sequence shown in FIG. 3 and identified as SEQ 4 and 14, and also the consensus framework identified in FIG. 3 as I21R33 and designated SEQ 7 and 17 makes an ideal framework sequence for the generation of bespoke intracellular antibodies.

In a preferred embodiment of this aspect of the invention, the antibody is an scFv. Preferred scFv's according to the invention include those comprising sequences shown in FIG. 3 and designated J38, 33, I21, I21R33, I21R33VHI21VL, Con 33, I21R33 (VHC22S,C92S).

Features of scFv's According to the Present Invention

Sequence analysis demonstrates that all anti-RAS scFv belong to VH3 and Vκ1 subgroup defined from Kabat database (Kabat et al., 1991) or IGHV3 and IGK1 subgroup defined from IMGT database (Lefranc and Lefranc, 2001). Most ICAb scFv selected which bind the antigens BCR or ABL (Tse et al., 2002) and TAU (Visintin et al., 2002), also belong to same subgroup, supporting the notion these subgroups of VH and Vκ framework can function intracellularly. This observation allowed a consensus framework scaffold to be defined (Tse et al., 2002). In this screening for anti-RAS scFv, one scFv (I21) shows high yields in bacteria periplasm, high solubility in mammalian cells but poor affinity of interaction in mammalian cells whilst other scFv (scFv33 and J48) have high affinity but relatively low yields of soluble expressed protein. The I21 scFv framework sequence however conforms closely with the consensus framework (Tse et al., 2002) in both the VH and VL regions and in support of the utility of this consensus, the present inventors found that mutation of scFv33 to the consensus framework (con33) or to the I21 framework (I21R33) improved this function including solubility and binding. Finally, when the scFv33 was mutated to the I21R consensus framework, retaining the scFv33 CDR sequences, the ICAbs were able to perform the crucial biological function of inhibiting activated RASG12V transformation of NIH3T3 cells. This is presumably due to the interaction of ICAb with the RAS target antigen (see FIG. 2). This illustrates the versatility of our approach in generating effective ICAbs for mammalian cell use.

The Requirement of Antibodies According to the Invention to Comprise Intradomain Disulphide Bridge Formation

The inventors have found that in order for anti activated RAS antibodies to form intracellularly functioning antibodies (that is antibodies which bind specifically to activated RAS within an intracellular environment and are both soluble and stable within such an environment) then intradomain disulphide formation is not required. That is an antibody molecule comprising both light and heavy chains (for example scFv) or a single domain type only (for example an Idab) wherein at least one of the cysteine residues which are normally present at positions 22 and 92 according to the Kabat nomenclature is no longer present or has undergone mutation to another residue, for example serine, is capable of binding specifically to activated RAS within an intracellular environment.

In particular, an antibody comprising both light and heavy chains wherein the framework region of the antibody is that represented by SEQ 10 and SEQ 20 respectively is capable of specifically binding to activated RAS within an intracellular environment.

Advantageously, an antibody according to the invention comprising both light and heavy chains wherein the framework region of the antibody is that represented by SEQ 10 and SEQ 20 or the framework region of the antibody is the same as that represented by SEQ 10 and 20 except that one or more of residues 22 and 92 are not cysteine residues (according to Kabat numbering).

(ii) Single Variable Domain Only Antibodies According to the Invention.

Using the IAC approach the present inventors have found that antibodies comprising heavy chain variable domains and not light chain variable domain (referred to as herein as heavy chain variable domain only antibodies) are capable of specifically binding to activated RAS within an intracellular environment. In addition, they have also found that antibodies comprising only light chain variable domain are capable of specifically binding to activated RAS within an intracellular environment.

Thus, antibodies comprising heavy chain variable domains and not light chain variable domains wherein the framework region sequences of the heavy chain variable domain are selected from the group shown in FIG. 3 and designated Con (SEQ 1) and I21R33 (SEQ 7) and the CDRs are selected from the group shown in FIG. 3 and designated J48 (SEQ 2) and 33 (SEQ 3) are capable of specifically interacting with activated RAS within an intracellular environment.

Moreover, antibodies comprising light chain variable domains and not heavy chain variable domains' wherein the framework region sequences of the light chain variable domain are selected from the group shown in FIG. 3 and designated Con (SEQ 11) and I21R33 (SEQ 17) and the CDRs are selected from the group shown in FIG. 3 and designated J48 (SEQ 12) and 33 (SEQ 13) are capable of specifically interacting with activated RAS within an intracellular environment.

According to the above aspect of the invention preferably the single domain type antibody is one comprising a single heavy chain variable domain attached to a bulking group (heavy chain variable domain IDab).

According to the present invention, IDabs capable of specifically binding to RAS do not require the presence of an intradomain disulphide bridge.

(a) The Requirement of Antibodies Comprising Heavy Chain Variable Domains Only According to the Invention for Intradomain Disulphide Bridge Formation.

The inventors have found that an antibody molecule according to the invention comprising one or more heavy chain variable domain only wherein at least one of the cysteine residues which are normally present at positions 22 and 92 in that domain according to the Kabat nomenclature is no longer present or has undergone mutation to another residue, for example serine, is capable of binding specifically to activated RAS within an intracellular environment.

According to the above aspect of the invention preferably the heavy chain only variable domain antibody is one comprising a single heavy chain variable domain (single heavy domain antibody).

(iii) Anti-RAS IDabs According to the Invention Isolated Using a Modified IAC Approach.

The inventors have used a modified IAC approach using an intracellular single variable domain (IDab) format, based on a previously characterised intrabody consensus scaffold, to generate diverse intrabody libraries for direct in vivo screening. Using this approach IDabs specific for RAS were isolated. These IDabs have an affinity for antigen binding of between 20 and 200 nM. Moreover, these IDabs were found to inhibit RAS-dependent oncogenic transformation of NIH3T3 cells.

The purpose of using intracellular antibodies is to bind to target proteins in vivo and elicit a biological response. The present inventors have shown herein that single domains (in this case, VH alone but VL alone should possess the same property) can be effective intracellular reagents showing excellent solubility and stability, and thus are ideal for binding specifically and with high affinity to antigen in vivo.

Several considerations make single domains attractive as intrabodies compared to scFv and other formats. The association of VH and VL domains is weak in scFv (Glockshuber et al., 1990) and the dissociated form can be predominant becoming a target for aggregation and proteolysis inside cells. An alternative form of VH-VL heterodimer is the disulphide-stabilised Fv fragment (dsFv) (Reiter & Pastan, 1996), but this in not a good option for intrabodies because the disulphide bond is not formed inside cells. Natural H chain antibodies are found in camel and related species in the absence of light chains and these are effective for binding and specificity in vitro (reviewed in (Muyldermans et al., 2001)). In vitro VH libraries have been described (Davies & Riechmann, 1995; Davies & Riechmann, 1996; Reiter et al., 1999) in which the VL interface of the VH domain was mutated to mimic the camel VH domain (Davies & Riechmann, 1995; Davies & Riechmann, 1996; Muyldermans et al., 1994). Camelisation of the VH framework of the anti-RAS VH IDabs (mutations G44E, L45R, W47G or W471 (Davies & Riechmann, 1995; Davies & Riechmann, 1996)) destroyed antigen binding activity in vivo as judged by the CHO luciferase reporter assay (data not shown). However, the IDabs described here, based on the IAC consensus scaffold, are expressed well as soluble proteins in cells and non-specific interactions with non-relevant antigen have not been detected. This suggests that modifications may not be useful for IDab intrabody applications. Rather, the employment of pre-defined immunoglobulin framework scaffolds is likely to be more useful, as these can exhibit properties attuned to the intracellular environment (Tanaka & Rabbitts, 2003). IDabs based on the IAC consensus (Tanaka & Rabbitts, 2003; Tse et al., 2002) fulfil this prerequisite for function. Thus, single domain intracellular antibodies are the smallest antibody fragment known at present with potential for in cell use.

A robust, rapid and simple procedure is needed to identify effective intrabodies and the present inventors IAC² approach has taken advantage of direct screening in the in vivo milieu to facilitate the isolation of those intrabodies which can fold adequately, have sufficient stability and can function in vivo. Specially designed, diverse intrabody libraries have an advantage for this objective as the process of deriving antigen-specific intrabodies would be greatly simplified and success more likely. The single domain intrabody format provides the means to achieve this since libraries using the scFv format are limited by the complexity of the VH and VL combination where there are six random CDR loops. The maximum diversity using single domain libraries (only three CDR loops) is lower than scFv. Moreover, IDabs composed of monomeric domains may be advantageous for interaction with antigen as the contact area between antigen and antibody occurs over a small area which could target small, hidden epitopes not accessible conventional scFv intrabodies. In these settings, IDabs might, for instance, recognise clefts formed by fusion of two protein domains which can result from chromosomal translocations in cancers.

CDR Sequences Which Confer Upon Intracellularly Antibodies Comprising them the Ability to Bind Activated RAS Within an Intracellular Environment

The inventors have found that the CDR sequences of the activated RAS antibodies shown in FIG. 3 and depicted SEQ NO: 1a, b and c; SEQ No 2 a, b and c; SEQ No 3, a, b, c; SEQ 11 a, b, c; SEQ 12 a, b, c, SEQ 13 a, b, c; SEQ No 21 a, b and c; SEQ No 22 a, b and c; SEQ No 23 a, b and c; SEQ No 24 a, b and c; SEQ No 25 a, b and c; SEQ No 26 a, b and c; SEQ No 27 a, b and c; SEQ No 28 a, b and c; SEQ No 29 a, b and c, when attached to their respective heavy or light chain variable domain framework regions amino acid sequences to generate an intracellularly functional antibody, confer upon the resultant antibody the ability to selectively bind to activated RAS within an intracellular environment.

Advantageously, the CDRs are those selected from the group consisting of J48 and 33 as shown in FIG. 3.

Use of Intracellularly Functioning Anti Activated RAS Antibodies According to the Present Invention in the Treatment of RAS Associated Cancer.

The inventors have found that the ant-RAS antibodies isolated using IAC technology are activated RAS specific. That is they selectively bind to the activiated/activated form of RAS and not the non-activated form of RAS.

This finding allows intracellularly functioning anti activated RAS antibodies according to the present invention to be used in the treatment of RAS associated cancer and RAS associated transformation of cells. In addition, it permits antibodies according to the present invention to be used in the preparation of a medicament of the treatment of RAS associated cancer.

Anti-activated RAS antibodies according to the present invention for use in the treatment of cancer may comprise heavy chain and light chain variable domains or solely heavy chain variable domains and no light chain variable domains, for example a Dab.

(a) Structure of Antibodies According to the Present Invention for Use in the Treatment of Cancer.

Framework Sequences

The heavy chain variable domains of the antibodies of the invention comprise any one of the heavy chain framework region amino acid sequences selected from the group shown in FIG. 1 and designated Con and I21R33 and depicted as SEQ 1, and 7 respectively. In addition, anti-activated RAS antibodies according to the invention suitable for the treatment of cancer may comprise any one of those sequences listed above but in which one or both cysteines at position 22 and 92 has been replaced by a different amino acid such that a heavy chain variable domain intradomain disulphide bridge cannot form.

Preferred heavy variable chain framework sequences are those selected from the group consisting of SEQ 1 (Consensus) and SEQ 7 (I21R33) as shown in FIG. 3.

In addition, an activated RAS specific antibody according to the present invention may comprise one or more light chain variable domains. Framework regions for activated RAS antibodies to be used in the treatment of cancer are shown in FIG. 3 and are any of those selected from the group consisting of Con, I21R33 and depicted as SEQ 11 and 17 respectively in FIG. 3.

Preferred variable light chain framework sequences are those selected from the group consisting of SEQ 1 (Consensus) and SEQ (I21R33) as shown in FIG. 3.

CDR Sequences

According to the invention those variable domain CDRs selected from those amino acid sequences shown in FIG. 3 and depicted SEQ NO: 1a, b and c; SEQ No 2 a, b and c; SEQ No 3,a, b, c; SEQ 11 a, b, c; SEQ 12a, b, c, SEQ 13 a, b, c; SEQ No 21a, b and c; SEQ No 22 a, b and c; SEQ No 23 a, b and c; SEQ No 24 a, b and c; SEQ No 25 a, b and c; SEQ No 26 a, b and c; SEQ No 27 a, b and c; SEQ No 28 a, b and c; SEQ No 29 a, b and c and which when attached to their respective heavy or light chain variable domain framework regions amino acid sequences to generate an intracellularly functional antibody, confer upon the resultant antibody the ability to selectively bind to activated RAS within an intracellular environment.

Therefore the antibodies for use in the treatment of cancer preferably possess framework sequences listed above combined with the corresponding (that is light or heavy chain) CDR set selected from the group listed above.

Preferred antibodies for use in the treatment of cancer are antibodies, in particular IDabs or scFv having a framework sequence of I21R33 or the consensus sequence combined with CDR sequences of J48 or 33 as shown in FIG. 3. Such an antibody may be heavy chain variable domain only or comprise both light and heavy chains. Most advantageously the antibody is an scFv or a Dab as herein defined.

(b) Delivery of Antibodies to Cells

In order to introduce antibodies according to the present invention into an intracellular environment, cells are advantageously transfected with nucleic acids which encode the antibodies.

Nucleic acids encoding antibodies can be incorporated into vectors for expression. As used herein, vector (or plasmid) refers to discrete elements that are used to introduce heterologous DNA into cells for expression thereof. Selection and use of such vehicles are well within the skill of the artisan. Many vectors are available, and selection of appropriate vector will depend on the intended use of the vector, the size of the nucleic acid to be inserted into the vector, and the host cell to be transformed with the vector. Each vector contains various components depending on its function and the host cell for which it is compatible. The vector components generally include, but are not limited to, one or more of the following: an origin of replication, one or more marker genes, an enhancer element, a promoter, a transcription termination sequence and a signal sequence.

Moreover, nucleic acids encoding the antibodies according to the invention may be incorporated into cloning vectors, for general manipulation and nucleic acid amplification purposes.

Both expression and cloning vectors generally contain nucleic acid sequence that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of bacteria, yeast and viruses. The origin of replication from the plasmid pBR322 is suitable for most Gram-negative bacteria, the 2m plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into yeast or mammalian cells even though it is not capable of replicating independently of the host cell chromosome. DNA may also be replicated by insertion into the host genome. However, the recovery of genomic DNA is more complex than that of exogenously replicated vector because restriction enzyme digestion is required to excise the nucleic acid. DNA can be amplified by PCR and be directly transfected into the host cells without any replication component.

Advantageously, an expression and cloning vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

As to a selective gene marker appropriate for yeast, any marker gene can be used which facilitates the selection for transformants due to the phenotypic expression of the marker gene. Suitable markers for yeast are, for example, those conferring resistance to antibiotics G418, hygromycin or bleomycin, or provide for prototrophy in an auxotrophic yeast mutant, for example the URA3, LEU2, LYS2, TRP1, or HIS3 gene.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both an E. coli replication origin and an E. coli genetic marker conferring resistance to antibiotics, such as ampicillin.

Suitable selectable markers for mammalian cells are those that enable the identification of cells expressing the desired nucleic acid, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked nucleic acid. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the desired nucleic acid. Such a promoter may be inducible or constitutive. The promoters are operably linked to the nucleic acid by removing the promoter from the source DNA and inserting the isolated promoter sequence into the vector. Both the native promoter sequence and many heterologous promoters may be used to direct amplification and/or expression of nucleic acid encoding the antibody. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Promoters suitable for use with prokaryotic hosts include, for example, the β-lactamase and lactose promoter systems, alkaline phosphatase, the tryptophan (trp) promoter system and hybrid promoters such as the tac promoter. Their nucleotide sequences have been published, thereby enabling the skilled worker operably to ligate them a desired nucleic acid, using linkers or adaptors to supply any required restriction sites. Promoters for use in bacterial systems will also generally contain a. Shine-Delgarno sequence operably linked to the nucleic acid.

Preferred expression vectors are bacterial expression vectors which comprise a promoter of a bacteriophage such as phagex or T7 which is capable of functioning in the bacteria. In one of the most widely used expression systems, the nucleic acid encoding the fusion protein may be transcribed from the vector by T7 RNA polymerase (Studier et al, Methods in Enzymol. 185; 60-89, 1990). In the E. coli BL21(DE3) host strain, used in conjunction with pET vectors, the T7 RNA polymerase is produced from the λ-lysogen DE3 in the host bacterium, and its expression is under the control of the IPTG inducible lac UV5 promoter. This system has been employed successfully for over-production of many proteins. Alternatively the polymerase gene may be introduced on a lambda phage by infection with an int-phage such as the CE6 phage which is commercially available (Novagen, Madison, USA). other vectors include vectors containing the lambda PL promoter such as PLEX (Invitrogen, NL), vectors containing the trc promoters such as pTrcHisXpress™ (Invitrogen) or pTrc99 (Pharmacia Biotech, SE), or vectors containing the tac promoter such as pKK223-3 (Pharmacia Biotech) or PMAL (new England Biolabs, Mass., USA).

Suitable promoting sequences for use with yeast hosts may be regulated or constitutive and are preferably derived from a highly expressed yeast gene, especially a Saccharomyces cerevisiae gene. Thus, the promoter of the TRP1 gene, the ADHI or ADHII gene, the acid phosphatase (PH05) gene, a promoter of the yeast mating pheromone genes coding for the a- or α-factor or a promoter derived from a gene encoding a glycolytic enzyme such as the promoter of the enolase, glyceraldehyde-3phosphate dehydrogenase (GAP), 3-phospho glycerate kinase (PGK), hexokinase, pyruvate decarboxylase, phosphofructokinase, glucose-6-phosphate isomerase, 3-phosphoglycerate mutase, pyruvate kinase, triose phosphate isomerase, phosphoglucose isomerase or glucokinase genes, the S. cerevisiae GAL 4 gene, the S. pombe nmt 1 gene or a promoter from the TATA binding protein (IBP) gene can be used. Furthermore, it is possible to use hybrid promoters comprising upstream activation sequences (UAS) of one yeast gene and downstream promoter elements including a functional TATA box of another yeast gene, for example a hybrid promoter including the UAS(s) of the yeast PH05 gene and downstream promoter elements including a functional TATA box of the yeast GAP gene (PH05-GAP hybrid promoter). A suitable constitutive PH05 promoter is e.g. a shortened acid phosphatase PH05 promoter devoid of the upstream regulatory elements (UAS) such as the PH05 (−173) promoter element starting at nucleotide-173 and ending at nucleotide-9 of the PH05 gene.

Gene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from promoters normally associated with immunoglobulin sequences.

Transcription of a nucleic acid by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the vector at a position 5′ or 3′ to the desired nucleic acid, but is preferably located at a site 5′ from the promoter.

Advantageously, a eukaryotic expression vector may comprise a locus control region (LCR). LCRs are capable of directing high-level integration site independent expression of transgenes integrated into host cell chromatin, which is of importance especially where the gene is to be expressed in the context of a permanently-transfected eukaryotic cell line in which chromosomal integration of the vector has occurred.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions contain nucleotide segments transcribed as polyadenylated fragments in the untranslated portion of the mRNA encoding the immunoglobulin or the target.

Particularly useful for practising the present invention are expression vectors that provide for the transient expression of nucleic acids in mammalian cells. Transient expression usually involves the use of an expression vector that is able to replicate efficiently in a host cell, such that the host cell accumulates many copies of the expression vector, and, in turn, synthesises high levels of the desired gene product.

Construction of vectors according to the invention may employ conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing gene product expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe which may be based on a sequence provided herein. Those skilled in the art will readily envisage how these methods may be modified, if desired.

Antibodies may be directly introduced to the cell by microinjection, or delivery using vesicles such as liposomes which are capable of fusing with the cell membrane. Viral fusogenic peptides are advantageously used to promote membrane fusion and delivery to the cytoplasm of the cell.

Preferably, the immunoglobulin is fused or conjugated to a domain or sequence from such a protein responsible for translocational activity. Preferred translocation domains and sequences include domains and sequences from the HIV-1-trans-activating protein (Tat), Drosophila Antennapedia homeodomain protein and the herpes simplex-1 virus VP22 protein. By this means, the immunoglobulin is able to enter the cell or its nucleus when introduced in the vicinity of the cell.

Exogenously added HIV-1-trans-activating protein (Tat) can translocate through the plasma membrane and to reach the nucleus to transactivate the viral genome. Translocational activity has been identified in amino acids 37-72 (Fawell et al., 1994, Proc. Natl. Acad. Sci. U.S.A. 91, 664-668), 37-62 (Anderson et al., 1993, Biochem. Biophys. Res. Commun. 194, 876-884) and 49-58 (having the basic sequence RKKRRQRRR) of HIV-Tat. Vives et al. (1997), J Biol Chem 272, 16010-7 identified a sequence consisting of amino acids 48-60 (CGRKKRRQRRRPPQC), which appears to be important for translocation, nuclear localisation and trans-activation of cellular genes. Intraperitoneal injection of a fusion protein consisting of -galactosidase and a HIV-TAT protein transduction domain results in delivery of the biologically active fusion protein to all tissues in mice (Schwarze et al., 1999, Science 285, 1569-72)

The third helix of the Drosophila Antennapedia homeodomain protein has also been shown to possess similar properties (reviewed in Prochiantz, A., 1999, Ann N Y Acad Sci, 886, 172-9). The domain responsible for translocation in Antennapedia has been localised to a 16 amino acid long peptide rich in basic amino acids having the sequence RQIKIWFQNRRMKWKK (Derossi, et al., 1994, J Biol Chen, 269, 10444 50). This peptide has been used to direct biologically active substances to the cytoplasm and nucleus of cells in culture (Theodore, et al., 1995, J. Neurosci 15, 7158-7167). Cell internalisation of the third helix of the Antennapedia homeodomain appears to be receptor-independent, and it has been suggested that the translocation process involves direct interactions with membrane phospholipids (Derossi et al., 1996, J Biol Chem, 271, 18188-93).

The VP22 tegument protein of herpes simplex virus is capable of intercellular transport, in which VP22 protein expressed in a subpopulation of cells spreads to other cells in the population (Elliot and O'Hare, 1997, Cell 88, 223-33). Fusion proteins consisting of GFP (Elliott and O'Hare, 1999, Gene Ther 6, 149-51), thymidine kinase protein (Dilber et al., 1999, Gene Ther 6, 12-21) or p53 (Phelan et al., 1998, Nat Biotechnol 16, 440-3) with VP22 have been targeted to cells in this manner.

Particular domains or sequences from proteins capable of translocation through the nuclear and/or plasma membranes may be identified by mutagenesis or deletion studies. Alternatively, synthetic or expressed peptides having candidate sequences may be linked to reporters and translocation assayed. For example, synthetic peptides may be conjugated to fluoroscein and translocation monitored by fluorescence microscopy by methods described in Vives et al. (1997), J Biol Chem 272, 16010-7. Alternatively, green fluorescent protein may be used as a reporter (Phelan et al., 1998, Nat Biotechnol 16,440-3).

Any of the domains or sequences or as set out above or identified as having translocational activity may be used to direct the immunoglobulins into the cytoplasm or nucleus of a cell. The Antennapedia peptide described above, also known as penetratin, is preferred, as is HIV Tat. Translocation peptides may be fused N-terminal or C-terminal to single domain immunoglobulins according to the invention. N-terminal fusion is preferred.

Also of use for the delivery of antibodies to cells is the TLM peptide. The TLM peptide is derived from the Pre-S2 polypeptide of HBV. See Oess S, Hildt E Gene Ther 2000 May 7:750-8. Anti-DNA antibody technology is also of use. Anti-DNA antibody peptide technology is described in Alexandre Avrameas et al., PNAS val 95, pp 5601-5606, May 1998; Therese Ternynck et al., Journal of Autoimmunity (1998) 11, 511-521; and Bioconjugate Chemistry (1999), vol 10 Number 1, pp 87-93.

Further Uses of Antibodies According to the Present Invention.

Antibody molecules according to the present invention, preferably scFv molecules may be employed in in vivo therapeutic and prophylactic applications, in vitro and in vivo diagnostic applications, in vitro assay and reagent applications, in functional genomics applications and the like.

Therapeutic and prophylactic uses of antibodies and compositions according to the invention involve the administration of the above to a recipient mammal, such as a human. Preferably they involve the administration to the intracellular environment of a mammal.

Substantially pure antibodies of at least 90 to 95% homogeneity are preferred for administration to a mammal, and 98 to 99% or more homogeneity is most preferred for pharmaceutical uses, especially when the mammal is a human. Once purified, partially or to homogeneity as desired, the immunoglobulin molecules may be used diagnostically or therapeutically (including extracorporeally) or in developing and performing assay procedures using methods known to those skilled in the art.

In the instant application, the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

The selected antibodies molecules of the present invention can perturb activated RAS protein function in vivo and thus will typically find use in preventing, suppressing or treating cancer. Using this approach, cells carrying the RAS oncoprotein could be specifically killed, sparing the normal ones.

Animal model systems which can be used to screen the effectiveness of the selected antibodies of the present invention in protecting against or treating disease are available. Suitable models of cancer will be known to those skilled in the art.

Generally, the selected antibodies of the present invention will be utilised in purified form together with pharmacologically appropriate carriers. Typically, these carriers include aqueous or alcoholic/aqueous solutions, emulsions or suspensions, any including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The selected antibodies of the present invention may be used as separately administered compositions or in conjunction with other agents. These can include various immunotherapeutic drugs, such as cylcosporine, methotrexate, adriamycin or cisplatinum, and immunotoxins. Pharmaceutical compositions can include “cocktails” of various cytotoxic or other agents in conjunction with antibodies of the present invention or even combinations of the antibodies, according to the present invention.

The route of administration of pharmaceutical compositions according to the invention may be any of those commonly known to those of ordinary skill in the art. For therapy, including without limitation immunotherapy, the selected antibodies of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. The dosage and frequency of administration will depend on the age, sex and condition of the patient, concurrent administration of other drugs, counterindications and other parameters to be taken into account by the clinician.

The selected antibodies of the present invention can be lyophilised for storage and reconstituted in a suitable carrier prior to use. Known lyophilisation and reconstitution techniques can be employed. It will be appreciated by those skilled in the art that lyophilisation and reconstitution can lead to varying degrees of functional activity loss and that use levels may have to be adjusted upward to compensate.

The compositions containing the present selected antibodies of the present invention or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected immunoglobulin per kilogram of body weight, with doses of 0.05 to 2.0 mg/kg/dose being more commonly used. For prophylactic applications, compositions containing the present selected immunoglobulin molecules or cocktails thereof may also be administered in similar or slightly lower dosages.

A composition containing one or more selected antibody molecules according to the present invention may be utilised in prophylactic and therapeutic settings to aid in the alteration, inactivation, killing or removal of a select target cell population in a mammal. In addition, the selected repertoires of polypeptides described herein may be used extracorporeally or in vitro selectively to kill, deplete or otherwise effectively remove a target cell population from a heterogeneous collection of cells. Blood from a mammal may be combined extracorporeally with the selected antibodies, cell-surface receptors or binding proteins thereof whereby the undesired cells are killed or otherwise removed from the blood for return to the mammal in accordance with standard techniques.

The invention is further described, for the purposes of illustration only, in the following examples.

### EXAMPLES

### Example 1

Materials and Methods-IAC Approach

Ras Antigen

Recombinant activated HRAS (G12V; residues 1-166) was expressed in bacterial cells harbouring expression plasmids based on pET11a (Novagen) and purified by ion-exchange chromatography and gel-filtration described elsewhere (Pacold et al., 2000). To prepare the active form of RAS antigen, 3 mg of purified HRASG12V protein was loaded with 2 mM of 5′-guanylylimidodi-phosphate (GppNp, Sigma), non-hydrolysable analogue of GTP, using the alkaline phosphatase protocol (Herrmann et al., 1996). This GppNp-bound HRASG12V was used as antigen throughout.

In Vitro scFv Phage Library Screening and Preparation of Specific scFv-VP16 Yeast Library.

The IAC screening of three different scFv libraries (de Wildt et al., 2000; Sheets et al., 1998) were performed as described (Tse et al., 2000; Tse et al., 2002) (see also a link within the Laboratory of Molecular Biology website http://mrc-lmb.cam.ac.uk) with slight modifications. In outline, a first panning step used phage Ab library screens was performed using 50 μg/ml HRASG12V antigen in PBS containing 1 mM MgCl₂, bound to immunotubes. Anti-RAS bound phage were rescued and amplified in E. coli TG1. The scFv DNA fragments were sub-cloned into the pVP16 yeast vector and 4.13×10⁶ clones used for yeast screening. The RAS bait was prepared by cloning truncated HRASG12V cDNA into the EcoR1-BaniH1 site of pBTM116 vector. The pBTM116-RASG12V bait vector (tryp+) was transfected into S. cerevisiae L40 using the lithium acetate/polyethylene glycol method (Tse et al., 2000), and colonies growing on Trp-plates were selected. The expression of LexA-RAS fusion protein was confirmed by Western blot using anti-pan RAS (Ab-3, Oncogene Research Product). For library screening, 100 μg of yeast scFv-VP16 library DNA were transformed into L40 clone stably expressing antigen. Positive colonies were selected for His prototropy and confirmed by β-galactosidase (β-gal) activity by filter assay. For the isolated individual clones, false positive clones were eliminated and true positive clones were confirmed by re-testing of His independent growth and β-gal activation.

Purification of scFv for In Vitro Assay

Periplasmic bacterial expression of scFv was as described (Tse et al., 2000). The scFv were cloned into pHEN2 vector (see www.mrc-cpe.cam.ac.uk for map) and expressed for 2 hours at 30° C. with 1 mM ITPG in 1 litre culture of E coli HB2151 cells. The cells were harvested and extracted periplasmic proteins with TES buffer (Tris-HCl (pH 7.5), EDTA, sucrose). The periplasmic proteins were dialyzed overnight against 2.5 litre of PBS including 10 mM imidazole. Immobilised metal ion affinity chromatograpy of periplasmic scFv was carried out at 4° C. for 1 hour with 4 ml of Ni-NTA agarose (QIAGEN). The agarose was washed 4 times with 20 ml of PBS with 20 mM imidazole. The polyhistidine-tagged scFv were eluted with 4 ml of 250 mM imidazole in PBS. The eluate was dialyzed overnight against 2.5 liter of 20 mM Tris-HCl (pH 7.5) including 10% glycerol at 4° C. Purified scFv was concentrated to 1 to 5 mg/ml using Centricon concentrator (YM-10, Amicon) and the aliquots were stored at −70° C. Protein concentration of purified scFv were measured using Bio-Rad Protein assay Kit (Bio-Rad).

ELISA Assays

The ELISA plate wells were coated with 100 μl of purified HRASG12V-GppNp antigen (4 μg/ml, approximately 200 nM) in PBS overnight at 4° C. Wells were blocked with 3% bovine serum albumin (BSA)-PBS for 2 hours at room temperature.

The respective purified scFv (approximately 450 ng) were diluted in 90 μl in 1% BSA-PBS and allowed to bind for 1 hour at 37° C. After washing 3 times with PBS containing 0.1% Tween-20 (PBST), horseradish peroxidase (HRP) conjugated anti-polyhistidine (HIS-1, Sigma) monoclonal antibody which were diluted 1:2000 in 1% BSA-PBS were allowed to bind for 1 hour at 37° C. After washing 6 times with PBST, HRP activity was visualised using 3,3′,5,5-tetramethylbenzidine (TMB) liquid substrate system according to manufacturer's instruction. The reaction was stopped with 0.5M hydrosulphate and data collected with a microtiter plate reader (450˜650 nm filter). To verify the specificity of scFv with antigen, competitive ELISA assay was also performed. scFv were pre-incubated with HRASG12V-GppNp antigen (8 μg/ml) for 30 minutes at room temperature, before adding the mixture to the antigen coated ELISA wells. All measurements were performed in duplicate.

Surface Plasmon Resonance Analysis

The BIAcore 2000 (Pharmacia Biosensor) was used to measure the binding kinetics of scFv with antigen. To immobilise antigen on a CM5 sensorchip, the sensorchip was first activated by flowing 40 μl of the mixture of EDC/NHS(N-ethyl-N-(dimethylaminopropyl) carbodiimide hydrochloride/N-hydroxysuccinimide) at 10 μl/min flow rate. 100 μg/ml of purified HRASG12V-GppNp in 10 mM sodium acetate, pH 3.5 was injected and immobilised until approximately 1500 RU. After immobilisation, the chip was inactivated with 40 μl of ethanolamine-HCl. Purified scFv (10-500 nM) were loaded at flow rate of 20 μl/minute at 25° C. (running buffer HBS-EP (0.01 M HEPES, pH 7.4, 0.15M NaCl, 3 mM EDTA, 0.005% v/v polysorbate 20) plus 2 mM MgCl₂,) on 2 channels of the chip containing either immobilised HRASG12V-GppNp or no antigen, for the determination of the binding affinity of scFv. Each determination was performed in duplicate. The antigen immobilised surface on the sensorchip after binding scFv was regenerated by rinsing with 10 mM HCl until the starting baseline was achieved. The kinetic rate constants, kₒₙ and k_{off}, were evaluated using the BLAevaluation 2.1 software supplied by the manufacturer. Kd values were calculated from k_{off} and kₒₙ rate constants (Kd=k_{off}/kₒₙ).

Mammalian In Vivo Antigen-Antibody Interaction Assay.

The scFv were cloned into Sfi1 and Not1 site of pEF-BOS-VP16 expression vector (manuscript in preparation). The HRAS expression plasmid (pM1-RASG12V) expressing RASG12V in-frame with Gal4 DBD, was made by sub-cloning HRASG12V cDNA (codons 1-166) into EcoR1/BamH1 site of pM1 vector (Sadowski et al., 1992). The baits pM1/β-gal and the prey pEF-BOS-VP16/R4 (anti-β-gal scFv), used as positive or negative controls, have been described (Tse and Rabbitts, 2000). COS7 cells were transiently co-transfected with 500 ng of pG5-Luc reporter plasmid (de Wet et al., 1987), 50 ng of pRL-CMV (Promega), 500 ng of pEF-BOS-VP16/scFv and 500 ng of pM1/antigen bait with 8 μl of LipofectAMINE™ transfection reagent (Invitrogen, according to manufacture's instruction). Forty-eight hours after transfection, the cells were washed once with PBS and lysed in 500 μl of 1× passive lysis buffer (Promega) at room temperature for 15 min with gently shaking. 2011 of cell lysate was assayed using Dual-Luciferase Reporter Assay System (Promega) in a luminometer. Transfection efficiency was normalised with the Renilla luciferase activity. The fold luciferase activity was calculated by dividing the normalised Firefly luciferase activity of the sample containing the vector alone. The data represent two experiments performed in duplicate.

Immunofluorescence Assays

scFv DNA fragments were cloned into the NcoI-NotI site of pEF-nuc-myc (Invitrogen) with nuclear localisation signal (nls) at N-terminal and myc-tag at C-terminal of exprssed scFv. For expression of RAS antigen, full length RASG12V cDNA was cloned into the Kpn1-EcoR1 site of pHM6 vector (Boehringer Mannheim) to encode RAS with HA-tag at N-terminal and His6-tag at C-terminal. The day before transfection, 1.2×10⁴ COS7 cells were seeded on Lab-Tek II Camber slide (Nalge Nunc International). The plasmids were co-transfected using Lipofectamine and forty-eight hours after transfection, cells were washed twice with PBS, permeabilised with 0.5% Triton X in PBS and fixed with 4% paraformaldehyde in PBS. Cells were stained with anti c-myc mouse monoclonal antibody (Santa Cruz; 9E10) and anti-HA rabbit polyclonal serum (Santa Cruz; sc-805) both at dilutions of 1:100. Secondary antibodies, fluorescein-linked sheep anti-mouse antibody and Cy3-linked goat anti-rabbit antibody (Amersham Pharmacia Biotech (APB)), were used at dilutions of 1:200 for staining. After several washes with PBS, the slides were overlaid with cover-slips and staining patterns were studied using a Bio-Radiance confocal microscope (Bio-Rad).

Western Blot Analysis

To evaluate the expression level and solubility of scFv in mammalian cells, the scFv or scFv-VP16 fusion proteins were expressed in COS7 cells. For scFv expression, scFv DNA fragments were cloned into Nco1/Not1 sites of pEF-myc-cyto expression vector (Invitrogen). The day before transfection, COS7 cells were seeded at about 2×10⁵ per well in 6 well culture plate (Nunc). 1 μg of pEF-myc-cyto-scFv or pEF-BOS-scFv-VP16 were transiently transfected with 8 μl of LipofectAMINE. 48 hours after transfection, the cells were washed once with PBS, lysed for 30 minutes in ice cold extraction buffer (10 mM HEPES, pH 7.6, 250 mM NaCl, 5 mM EDTA, 0.5% NP40, 1 μg/ml leupeptin, 1 μg/ml pepstatin A, 0.1 mg/ml aprotinin, 1 mM phenylmethanesulsonyl fluoride (PMSF)) and centrifuged for 10 minutes at 13,000 rpm at 4° C. The pellets (“insoluble” fraction) and the supernatants (“soluble” fraction) were analysed by SDS-PAGE, followed by Western blot using anti-myc (9E10) monoclonal antibody (for detection of scFv) or anti-VP16 (14-5, Santa-Cruz) monoclonal antibody (for scFv-VP16AD fusion) as primary antibody and HRP-conjugated rabbit anti-mouse IgG antibody (APB) as secondary antibody. The blots were visualised by enhanced chemiluminescence (ECL) detection kit (ABP)

Mutation of Framework Residues for Anti-RAS scFv.

pEF-BOS-VP16 with anti-RAS scFv33 was used for original templates and Pfu DNA polymerase was used throughout. The construct I21R33 (sequence shown in FIG. 3), which comprises FRs of anti-RAS scFvI21 and the CDRs of anti-RAS scFv33, was constructed using step-by-step site-specific mutagenesis of scFv33 as primary template using footprint mutagenesis (manuscript in preparation). I21R33 (VHC22S;C92S), con33 and I21R33VHI21VL (FIG. 3) were also constructed by mutations of I21R33 using footprint mutagenesis with appropriate oligonucleotides. All scFv constructs were digested with Sfi1 or Nco1, and Not1 and subcloned into pEF-BOS-VP16 (for in vivo antigen antibody interaction assay) and pEF-myc-cyto vector (for expression of scFv in mammalian cells). All mutated scFv constructs were verified by DNA sequencing.

Transformation Assays in NIH3T3 Cells

RAS protein is localised to the plasma membrane of cells and therefore to localise scFv to cell membrane we used the pEF-Memb vector (Invitrogen). The scFv expression plasmid was constructed by introducing carboxyl terminal 20 amino acid residues of HRAS into the Not1-Xba1 site of pEF-myc-cyto vector. This expression vector also was introduced FLAG-tag peptide (MDYKDDDDK) and alternative Sfi1 cloning site into blunt-ended Sfi1 site of pEF-Memb vector named pEF-FLAG-Memb. The scFv were sub-cloned into Sfi1-Not1 of pEF-FLAG-Memb. For expression of RASG12V, HRASG12V mutant cDNA were subcloned into expression vector pZIPneoSV(X) REF. Low passage NIH3T3 cells clone D4 (a gift from Dr Chris Marshall) were seeded at 2×10⁵ cells per well in 6-well plates the day before transfection, For transfection, 2 μg of each pEF-FLAG-Memb-scFv plus 100 ng of pZIPneoSV(X)-HRASG12V vector was used, using 12 μl of LipofectAMINE™. Two days later, the cells were transferred to 10 cm plates and grown for two weeks in DME medium containing 5% donor calf serum (Invitrogen) and penicillin and streptomycin. The plates were finally stained with crystal violet and the number of foci counted.

### Example 2

Isolation of Specific Intracellular Antibody Fragments which Recognise RAS Protein In Vivo

We have applied the intracellular antibody capture technique (Visintin et al., 1999) to the isolation of anti-RAS ICAbs. The sequential steps comprise initial in vitro phage scFv library panning with purified RAS protein and in vivo antigen-antibody two hybrid interaction screening to isolate specific intracellular antibodies. For in vitro phage Ab screen, purified carboxyl-terminal truncated human Ha-RASG12V was used as antigen, bound to 5′-guanylylimidodi-phosphate (GppNp, Sigma, non-hydrolysable analogue of GTP). After one round of in vitro panning, about 1.18×10⁶ antigen-bound phage were recovered from 2.7 X10³ initial phage (FIG. 1). The sub-library was prepared as phagemid DNA and cloned into a yeast VP16 transcriptional activation domain vector to make an anti-RAS scFv-VP16-AD library (about 4×10⁶ clones). This yeast sub-library was transfected into a yeast strain (L40 with his and β-gal reporter genes) expressing the fusion protein bait comprising the LexA-DBD fused to RAS-G12V. A total of approximately 8.45×10⁷ yeast colonies were screened (FIG. 1). 428 colonies grew in the absence of histidine and these clones also showed activation of β-gal. The scFv-VP16-AD plasmids were isolated from the histidine-independent, β-gal positive clones and assorted by their DNA restriction patterns. More than 90% of these scFv-VP16-AD plasmids had an identical DNA finger printing pattern and twenty were sequenced and found to have identical DNA sequences. Those scFv with differing DNA finger print patterns were co-transformed with the pBTM/RASG12V bait in fresh yeast and assayed for histidine-independent growth and β-gal activation. Three anti-RAS scFv, designated 33, J48 and 121, were thus identified (FIG. 1). The specificity of these scFv for binding to RAS in yeast was further verified by their lack of interaction with the LexA DBD (made from the empty pBTM116 vector) and a non-relevant antigen (β-galactosidase) (data not shown).

The efficacy of the anti-RAS ICAbs was confirmed using a mammalian cell reporter assay and in vivo antigen co-location assays (FIG. 2). The mammalian cell assay used was luciferase production from a luciferase reporter gene. The three scFv were shuttled into a mammalian expression vector, pEF-BOS-VP16, which has the elongation factor-1a promoter (Mizushima and Nagata, 1990) and the VP16 transcriptional activation domain (AD). The scFv were cloned in frame with the VP16 segment, on its N-terminal side (Triezenberg et al., 1988). The RASG12V antigen was cloned into the pM vector (Sadowski et al., 1992) which has the GAL4-DBD as an N-terminal fusion with antigen (pM-RASG12V). pEFBOSVP16-scFv and pM-RASG12V were co-transfected into COS7 cells with the luciferase reporter plasmid. More than 10-fold activation was observed when scFv33 or J48 ICAb-VP16 fusion were expressed with the bait antigen RASG12V (FIG. 2A) but none with a non-relevant antigen β-galactosidase. No activation was observed, however, when the yeast anti-RAS ICAb I21 was co-expressed with RASG12V bait (FIG. 2A). Similar results were obtained in other mammalian cell lines viz. Hela and CHO cells. The failure of ICAb I21 to detectably interact with antigen in this mammalian cell assay, as opposed to yeast, may simply be due to it having insufficient affinity or may reflect the relative insensitivity of mammalian compared to yeast assays, perhaps due to factors such as transfection efficiency, reporter gene activation requiring access to endogenous transcription factors and/or the expression level of antigen or antibody.

The observed interaction of scFv33 and J48 in a yeast system expressing lexA-DBD and a mammalian system expressing Gal4-DBD is a good indicator that the scFv interact with a native epitope of the RAS antigen, rather than an artificial one due to fusion of RAS and a DBD in the bait. Additional evidence for this was obtained from co-location assays in which the native RAS antigen was expressed together with the scFv to which nuclear localisation signals (nls) had been added. COS7 cells were co-transfected with a RAS expression vector with HA epitope tag and scFv expression vectors encoding scFv with a myc epitope tag. After 52 hours, RAS antigen was detected with anti-HA tag Ab and scFv with anti-myc tag Ab (FIG. 2B). When the RAS antigen was expressed alone or with a non-relevant ICAb (scFvR4 (Martineau et al., 1998)), the antigen was detected in the cytoplasm and antibody in the nucleus (FIG. 2B, lower panels), whereas if the antigen was co-expressed with the anti-RAS ICAb 33 with a nls, co-location of RAS antigen and scFv was observed in the nucleus. These means that the anti-RAS ICAbs 33 have sufficient expression and affinity to bind RAS antigen in vivo and cause re-location within the cell (similar results were found with anti-RAS scFv J48, data not shown).

### Example 3

The Sequence and Bacterial Expression of Anti-RAS scFv

The anti-RAS scFv (33, J48 and I21) were sequenced and derived protein sequence aligned (FIG. 3). All three scFv belong to VH3 subgroup joined to the JH5 and to the Vκ1 subgroup. Our previous data on anti-BCR and anti-ABL scFv (Tse et al., 2002), which were isolated only from the library of Sheets et al (Sheets et al., 1998), also belong to VH3 and Vκ1 subgroup. In our previous study we were able to define a consensus framework which was derived by comparing the anti-BCR and anti-ABL scFv (Tse et al., 2002) and an analogous study was conducted with anti-TAU ICAbs (Visintin et al., 2002). We concluded that a framework composed of VH3 and VKl is highly amenable for scFv function inside the cell and the consensus set a basic sequence on which to design other ICAbs. The anti-RAS ICAbs described here help to refine this concept.

The levels of expression of three anti-RAS scFv were initially examined by bacterial periplasmic expression. These scFv were sub-cloned into pHEN2, which has the PelB leader sequence 5′ to the scFv, allowing the periplasmic expression of soluble scFv protein (see www.mrc-cpe.cam.ac.uk for map). Periplasmic scFv extracts were purified by immobilised metal ion affinity chromatography (IMAC) and protein preparations separated by SDS-PAGE (FIG. 4). The scFvI21 accumulated mainly in the soluble fraction, when secreted to the periplasm at 30° C. and the periplasmic expression yield was approximately 3 mg per litre culture. The other anti-RAS scFv (33 and J48) were expressed at less than 0.1 mg per litre. Comparison of the anti-RAS scFv sequences with the consensus ICAb sequence (FIG. 3), reveals only four differences in the VH framework residues of 33 and J48, one of which is position 7 in VH FR1. This residue is one of three which influence conformation of this region (Jung et al., 2001) and may thus influence ICAb 33 and J48 solubility. I21 conforms to the consensus in positions VH FR16, 7 and 10.

### Example 4

Biochemical and Biophysical Characterisation of Anti-RAS scFv

The properties of the ICAbs isolated in our work were aslo characterised using two in vitro assays. The interaction of the scFv with RAS antigen was investigated with ELISA and biosensor assays using purified scFv made in bacteria. RASG12V-GppNp was coated as antigen onto ELISA plates, challenged with purified scFv and bound scFv was detected using HRP conjugated anti-His tag antibody (FIG. 5). All three anti-RAS scFv produced significant signals with RAS antigen compared with BSA and the signals were inhibited by pre-incubation with RASG12V antigen, as a measure of specificity of the interaction. These results further suggest that these anti-RAS scFv may interact only with the native form RASG12V-GppNp.

The affinities of binding anti-RAS scFv to antigen were measured by binding kinetics in the BIAcore (FIG. 6). The Kd of scFv33 and J48 were determined to be 1.39±1.31 nM, 3.63±0.15 nM (FIG. 6B). The affinity difference of their scFv may reflect the differences of CDR1 sequence in VH domain. The scFvI21 had a Kd of 2.16±0.25 μM, about three order of magnitude weaker than scFv33 or J48. This weak affinity of scFvI21, in the micromolar range, is consistent with its weak β-gal reporter gene activation in the yeast in vivo antigen-antibody interaction assay and lack of detectable binding in mammalian cell assays.

### Example 5

The Functional Improvement of Anti-RAS scFv by Modification of the scFv Framework Sequences

There is an excellent quantitative correlation between stability and yield of scFv when expressed in bacterial cells and mammalian cells, in which scFvI21 showed a higher expression yield in bacteria (FIG. 4) and in mammalian cell cytoplasm (FIG. 7), compared with the other two anti-RAS scFv. While all scFv tested in COS7 expression showed significant amounts of ‘insoluble’ scFv (FIG. 7B), the best expression levels were apparent for scFvI21 (FIG. 7A). The ability to improve solubility and stability of anti-RAS scFv33 in vivo, as well as in vitro, was assessed by mutating the framework of scFv33, to include some or all of the 13 amino acid differences between it and scFvI121 (FIG. 3). When scFv33 was mutated in the VH FR regions to make it equivalent to I21 (but including arg at the end of FR3 rather than lys), excellent in vivo solubility was found (FIG. 7A, I21R-33). In addition, mutation of both Cys residues, needed for intra-chain disulphide bonds, to Ser (FIG. 7, scFv I21R-33(VHC22S;C92S) had only a small effect on soluble expression levels.

The in vivo interaction of the various mutants of the scFv was assessed in COS7 cells using the luciferase reporter assay (FIG. 8). FIG. 8A shows expression and luciferase reporter data of various modifications of the scFv33 framework compared to levels with scFv33 itself or scFvR4 (anti β-gal negative control, (Martineau et al., 1998)) and scFvI21 which does not give significant luciferase activity. One notable mutation of scFv33 is Arg94Lys (numbering according to Kabat et al (Kabat et al., 1991), position 106 according to IMGT, Lefranc et al (Lefrane and Lefranc, 2001)) which completely eliminated reporter response (FIG. 8A) even though the expression of this scFv-VP16 is increased compared with original scFv 33 (FIG. 8A). The arginine residue at position 94 is very close to the antigen binding site (CDR3 of heavy chain) and may be involved in interaction with RAS antigen directly. Alternatively, the residue at this position may form a surface bridge across the CDR3 loop through its positively charged side chain with the carboxyl group of the aspartic acid at position H101 (Morea et al., 1998), and the substitution (Arg to Lys) may affect the critical conformation of CDR3. The other mutant scFv33 variants generally maintained their binding ability with RAS antigen as judged by the luciferase reporter assay (FIG. 8A). Interestingly, three mutant variants, VH(A74S+S77T), VL(I84T), and VH(Q1E+V5L+A7S+S28T)+VL(G100Q+L104V), were increased 1.5 to 2.5-fold in reporter gene activity, accompanied with an increase of scFv-VP16 in the soluble fraction.

The mutation of scFv33 into the framework of scFvI21 was performed, except arginine at position H94 was maintained (I21R33). Two further scFv33 variants were constructed, one in which scFv33 was converted to the ICAb consensus framework (con33) and one in which mutation of only the VH frameworks was carried out (I21R-33VHI21VL, FIG. 3). In the mammalian reporter assay, 2 to 3 fold-increase of reporter gene activity was observed with I21R33 and con 33, but with dramatically improved solubility compared with original scFv33 (FIG. 8B). These data show that the consensus, or I21, framework are most suitable scaffold for intracellular antibody expression and furthermore ICAb function can be improved using these frameworks.

### Example 6

Activity of Anti-RAS scFv Lacking Conserved Cysteine Residues In VH Domain.

The mutated anti-RAS scFv I21R33 interacts specifically with RAS antigen in COS7 cells, even though, in this reducing environment, scFv mostly cannot form disulphide bonds (Biocca et al., 1995; Tavladoraki et al., 1993). Perhaps a small population of over-expressed scFv does form disulphide bonds in the cytoplasm and interact with antigen in vivo, such as the anti-β galactosidase scFvR4, some of which is disulphide bounded in cytoplasm of bacteria (Martineau et al., 1998). Thus a small population could be detectable in vivo using our antigen-antibody interaction assay, if the scFv has a high affinity with antigen. However, in vitro studies have demonstrated that some scFv can be made which are disulphide-free but fold correctly (Proba et al., 1998; Worn and Pluckthun, 1998a). Therefore, to test the requirement for intra-chain S-S bonds, an expression vector encoding a mutant scFv lacking the cysteine residues at position 22 and 92 (Kabat numbering or 23 and 104 in IMGT numbering) was constructed. This scFv, based on the I21R33 sequence, had the two cys codons were mutated to serine (clone I21R33(VHC22S;C92S). A vector encoding this protein was tested in our mammalian reporter assay (FIG. 8B). The scFv protein was expressed at high levels and roughly comparable with those of I21R33 and I21 and the ability to activate the luciferase reporter was similar to the 12R33 scFv. These results show that anti-RAS scFv I21R33 can fold adequately without intra-chain disulphide bond and function inside cells in this condition.

### Example 7

Conversion of ICAbs Into Anti-RAS scFv to Block Tumorigenic Transformation

In the experiments discussed above, we sought to improve on the effectiveness of anti-RAS ICAbs by mutational analysis of the VH and VL framework regions to make them equivalent to canonical IAC consensus (Tse et al., 2002). A further test of the utility of our pre-determined consensus frameworks was carried out by assessing the ability of anti-RAS sequences to inhibit activated RAS transformation of NIH3T3 cells. We evaluated this by taking as a starting point the scFv the I21 clone which was isolated from the yeast screening (FIG. 1) using RAS as a bait but which does not have any significant activity in mammalian cells, despite being well expressed (FIG. 7). Mutatgenesis of the scFv33 to I21R33 (i.e. I21 framework with VH and VL CDRs of scFv33) gives a well expressed protein able to activate the luciferase reporter (FIG. 8B). We have used this in a competitive transformation assays in which NIH3T3 cells were transfected with a plasmid expressing activated HRAS alone (RASG12V) to yield transformed, foci (non-contact inhibited colonies) which can grow in multilayers and show a swirling appearance of spindle-shaped cells (FIG. 9A, RASG12V+empty scFv vector). When the NIH3T3 cells were co-transfected with the RASG12V vector together with one expressing scFvI21, essentially no difference to control was observed (FIGS. 9A and B) in keeping with the observed lack of activation of the RAS-dependent luciferase reporter assays. On the other hand, when RASG12V was expressed with the mutated I21 clone, scFvI21R33, the number of transformed foci reduced to 30% presumably due to interaction of the scFv with the RASG12V expressed protein and preventing its function. Thus the consensus scaffolds provide a basis for creation of functional scFv in our experiments.

### Example 8

Single Domain Antibody Fragments can Function as Intrabodies In Vivo

In the previous examples intracellular scFv antibodies were isolated by an intracellular antibody capture method (Tse et al., 2002) and their in vivo effectiveness for antigen binding was improved using step-by-step mutagenesis of the scFv framework to a consensus sequence (Tanaka et al., 2003).

We have now tested the ability of the individual domains of the anti-RAS scFv intrabodies (i.e. the single VH domain or the single VL domain) to bind antigen in vivo. Various expressed antibody fragments were tested in a luciferase reporter assay which comprised transfecting COS7 cells with a minimal luciferase reporter plasmid together with a vector encoding RAS antigen linked to the Gal4 DNA binding domain (DBD) and one encoding an antibody fragment linked to the VP16 transcriptional activation domain (AD). The expression of the intrabody-VP16 fusions was assessed by detection of proteins using Western blotting. All the clones support the expression of their respective proteins in COS7 cells and it is evident that both scFv and single domain intrabody fusions (VH and VL) are equivalently and well expressed.

The ability of the intrabodies to interact with their respective antigen in vivo was tested using a luciferase reporter gene assay. It is significant that the best luciferase activation was achieved with the anti-RAS VH single domain formats. For instance, the VH from intrabody anti-RAS scFv33 stimulates the reporter activity about 5 times more that the parental scFv clone). The anti-RAS VL single domain, however, did not activate at all (33VL). As we described previously (see Examples above) conversion of scFv33 to a consensus format (here we used the I21R33 version) had increased in vivo function in terms of antigen binding. The single domain VH derived from this intrabody also performed better than the parental molecule in this reporter assay. Finally, mutation of the cysteine residues, which are involved in the intra-domain disulphide bonds of the VH domains, had no substantial effect on in vivo expression or function (clones I21R33VH-C22S and I21R33VH-C92S). Thus single domain intrabodies (IDabs) can function without the intra-domain disulphide bond. We conclude that binding of the anti-RAS scFv33 to antigen can occur through the VH domain alone and an important corollary is that single domains appear to be excellent mediators of intracellular antibody function.

### Example 9

Direct Screening of Synthetic Single Domain Intracellular Antibody Libraries in Yeast-LAC² Approach.

The observed functioning of single VH domains in mammalian cells suggested that the IDab format could be generally useful for production of intracellular antibody libraries with sufficient diversity for isolation of antigen-specific IDabs directly by yeast antibody-antigen interaction procedures (Visintin et al., 1999). This idea was tested by generating IDab libraries, based on the previously described intrabody consensus framework (Tanaka & Rabbitts, 2003; Tse et al., 2002), for direct in vivo screening in yeast (Tse et al., 2000). Two IDab libraries were made by cloning diversified VH domains into the pVP16* vector to encode IDab-VP16 fusion proteins. The sizes of the libraries were around 3×10⁶ (IDab library 1) and 5×10⁷ (IDab library 2, estimated diversity ˜3×10⁷), which were complexities compatible with direct yeast screening.

The IDab libraries were screened with two different antigens (viz. HRASG12V and ATF-2) to ascertain their general utility. Yeast cells, which have his3 and lacZ reporter genes, were transfected the IDab libraries together with antigen bait clones encoding the antigen fused to the LexA DNA binding domain. In excess of a hundred clones showed histidine independent growth with either antigen bait (Table 1), suggesting the intracellular interaction of the antigen and VH single domain intrabodies. These clones were picked and assessed using a β-gal filter assay and the ten causing most rapid colour development were selected and sequenced. FIG. 3B shows the derived amino-acid sequences from the VH CDR regions, compared with the parental CDR regions of IDab 33. Among the selected clones, several identical sequences were found with IDabs selected against the different antigens suggesting that these clones bind with LexA DNA binding domain portion of the bait protein. This was assessed by re-assaying histidine-independent growth and β-gal activation of each IDab clone with the heterologous bait. In this way, we found that nine of the anti-RAS IDabs showed interaction not only with the cognate bait but also with the non-relevant ATF-2 bait (clones #1, #2, #4, #5, #8, #11, #14, #16, #19), consistent with these IDabs being anti-LexA intrabodies. The remaining ones were confirmed to have specificity against the RAS antigen. All the selected anti-ATF-2 IDabs were specific for the cognate antigen. Significant length variation of the VH CDR3 was found, especially in the anti-ATF-2 clones, consistent with the method of CDR3 randomisation, which included length variation from two to twelve codons.

### Example 10

Library Selected IDabs can Function in Mammalian Cells

Our results show that it is possible to select IDabs by directly screening a library in yeast (IAC² approach), thus avoiding the in vitro phage antibody library screening required in the original IAC method (Tse et al., 2000; Visintin et al., 2002). The efficacy of these IDabs in mammalian cells was tested using three different transcriptional transactivation assays. Firstly, we tested the IDab clones in the COS7 based luciferase reporter assay (Tanaka & Rabbitts, 2003). The IDab sequences were cloned into a mammalian expression vector to express the IDab fused with the VP16AD at the C-terminus. COS7 cells were transfected with IDab-VP16 constructs and either a specific bait expressing as a Gal4 DBD-antigen fusion or a bait comprising Gal4 DBD-LexA fusion. We observed a degree of variability in the activation of luciferase, with some clones giving a high stimulation of reporter activity, for instance anti-RAS clones #6 and #10, while some only produced a moderate stimulation, for instance anti-RAS clone #3 or the anti-ATF-2 clones #27 and #29. Interestingly, anti-RAS clone #3 not only has a long CDR3 compared to other anti-RAS IDabs (FIG. 3B), but only stimulated luciferase activation when co-expressed with HRAS, but not with KRAS and NRAS, whereas the anti-RAS IDab clones #6, #7, #9, #10, #12, #13, #17 and #18 stimulated luciferase activation when co-expressed with all three RAS antigens (data not shown). These data indicate that the anti-RAS intrabody #3 recognises a different epitope on the RAS molecule from the other IDabs. Clones #1, #2, #4, #11, #14, #16 and #19 stimulated significant reporter activity with LexA as a bait which, taken together with the finding that these IDabs bind both to LexA-RAS and LexA-ATF-2 baits, shows that they are anti-LexA DBD intrabodies.

Validation of mammalian cell activity of the anti-RAS IDabs was obtained using CHO cells which carry either chromosomal CD4 (Fearon et al., 1992) or GFP reporters. When these reporters are stimulated by transient expression of a complex between Gal4 DBD-antigen and IDab-VP16 fusion proteins, either the CD4 molecule in expressed at the surface of the CHO cells (CHO-CD4) or green fluorescent protein is produced in the cells (CHO-GFP). When a non-relevant intrabody, anti-β-gal scFvR4 (Martineau et al., 1998), was expressed with the RAS bait, no reporter activation was observed for either CHO-CD4 or CHO-GFP. However, around 20-40% of cells displayed CD4 or GFP expression when scFvR4 and a lacZ reporter were co-transfected. The bait specificity was reversed when anti-RAS IDab33 (the original IDab sub-cloned from the anti-RAS scFv33 (Tanaka & Rabbitts, 2003)) or anti-RAS IDab #6 or #10 were co-expressed with the baits, since activation was only observed with the RAS bait These results indicate that the yeast IDab library screening approach can select IDabs with sufficiently good in vivo properties to facilitate binding to relevant antigen within mammalian cells.

### Example 11

Single Domain Intracellular Antibodies are Expressed as Soluble Proteins In Vivo

The IDab intrabodies that we have used in these reporter assays are expressed as fusions with the VP16 activation domain and are well expressed. However, the VP16 domain of the fusion proteins could be a major determinant of solubility and stability in mammalian cells and it is possible that the single domains alone would not be well tolerated in vivo, as these antibody fragments do have a tendency to aggregate in vitro (Davies & Riechmann, 1995). Indeed, unmodified human VH domains, in the absence of the VL domain (i.e. with the hydrophobic VL interface exposed) are only monomeric at low protein concentrations in vitro and begin to aggregate as concentrations increase (Davies & Riechmann, 1995; Riechmann & Muyldermans, 1999). We have assessed IDab characteristics in vivo by expressing anti-RAS IDabs in NIH3T3 cells by transiently transfecting clones encoding either scFv or Dab antibody fragments and detection of expressed intrabodies by Western analysis using anti-FLAG tag antibodies. This expression analysis was performed in the presence or absence of antigen expression and proteins were extracted from detergent lysed cells either in the soluble fraction or as post-lysis insoluble material collected by centrifiugation. IDab and scFv intrabody proteins appeared in both cellular fractions in this analysis and no significant differences were observed whether or not antigen was co-expressed. Significantly, anti-RAS IDab clones #6 and #10 seemed to be expressed as soluble proteins better than scFv formats. These results suggest that IDabs can be more stable in cells than the scFv format, perhaps because scFv have a peptide linker, which may lead to proteolysis susceptibility, following poor association of VH and VL in vivo.

### Example 12

Single Domain Intracellular Antibodies Can Bind Antigen In Vitro with High Affinity

Determinants of ‘intracellular affinity’ in the complex milieu of the mammalian cell are binding affinity, expression levels and stability of the intrabody in presence and absence of antigen. It is not possible to determine binding affinity in vivo or to carry out studies of thermodynamic (or kinetic) stability or aggregation tendencies. However, to assess a parameter of intracellular affinity, we have determined the in vitro binding affinity of four selected anti-RAS IDab clones #3, #10, #12, compared to the original IDab 33. The Dab proteins were expressed in bacteria but the final yields of purified Dab proteins were rather low (up to 0.5 mg per 1 litre of culture), presumably because purification and concentration invokes the stickiness and aggregation of Dabs at high concentration in vitro (Riechmann & Muyldermans, 1999).

We measured the RAS antigen-binding affinities of the IDabs using a biosensor. The Kd of scFv33 was found to be about 10 nM (Table 2) which is consistent with our previous study (Tanaka & Rabbitts, 2003). The mutated scFvI21R33VHI21VL (in which the framework of anti-RAS scFv33 is mutated to the 121 consensus VH but retains the I21 VL sequence) maintains the affinity of scFv33 (Kd about 18 nM) consistent with the importance of the VH-antigen interaction. Loss of affinity was observed when the VH of scFv33 was made into the IDab format (Table 2; Kd of about 90 nM), being about one order of magnitude weaker than original scFv33. The Kd of anti-RAS IDab clones #3, #10, and #12 were around 180 nM, 120 nM, 26 nM, respectively. Thus, there is no obvious correlation between the in vitro affinity of the anti-RAS IDabs (which is a measure of real antigen-antibody interaction) and in vivo activity (where the total in vivo antigen-antibody interaction involves several factors). These data suggests that it is worthwhile evaluating IDabs in both in vivo and in vitro assays but nonetheless the binding affinity component of the selected IDabs is within a suitable range for in vivo function as antigen-binding moieties.

### Example 13

Oncogenic Transformation of NIH3T3 Cells can Inhibited by IDab Intrabodies

The purpose of intrabodies is to ablate or otherwise interfere with the function of proteins inside cells, for instance to block an abnormal function in a cancer cell. The function of oncogenic RAS is mediated through constitutive signalling in tumours and this can be emulated by introducing mutant RAS (HRASG12V; with a glycine to valine mutation at codon 12) into NIH3T3 cells, resulting in loss of contact inhibition and focus formation in confluent cell cultures. We have shown that scFv intrabodies, which have been selected by intracellular antibody capture, can inhibit the RAS-mediated transformation (Tanaka & Rabbitts, 2003). We have evaluated the utility of IDabs to inhibit transformation, by carrying out RAS transformation assays in the presence or absence of these antibody fragments (FIG. 10).

When an expression clone encoding mutant HRASG12V was transfected into NIH3T3 cells, growth of transformed, non-contact inhibited colonies was detected (FIG. 10A) whereas cells into which vector alone was introduced, retained their contact inhibition. This defined 100% and 0% relative transformation respectively (FIG. 10B). When the HRASG12V clone was co-transfected with scFvI21 (an scFv which has no detectable RAS binding in mammalian assays, although it is expressed efficiently (Tanaka & Rabbitts, 2003)), the transforming ability of the mutant RAS was unaffected, since the numbers of foci observed with HRASG12V alone or HRASG12V plus scFvI21 were approximately the same (FIGS. 10A and B). Conversely, we observed an ablation of transforming activity when HRASG12V was co-expressed with anti-RAS scFv (scFvI21R33VHI21VL in which the scFv comprises VH of anti-RAS scFv33 with VL of I21 (Tanaka & Rabbitts, 2003)), with only around 20% of focus formation compared with the HRASG12V control alone (FIG. 10B). Two anti-RAS IDabs were tested in this assay (IDab #6 and #10) which were chosen because of their excellent stimulation in the mammalian reporter assays and their good expression characteristics in NIH3T3 cells. These behaved in a similar way to the anti-RAS scFv, showing a dramatic effect on the transformation index. Anti-RAS IDab #6 and #10 reduced the transforming activity of oncogenic HRASG12V to below 10% of the transfected cells expressing HRASG12V alone (FIG. 10B). Thus, these IDabs can be expressed in mammalian cells and in sufficient quantity and quality to inhibit tumorigenic transformation. The data indicate that the IDab selection procedure will be generally useful for generating reagents with sufficiently good in vivo properties to interfere with protein function in mammalian cells.

### Example 14

Materials and Methods for the IAC² Approach.

Plasmids

Reporter Clones:—

The reporter plasmids pG5-Luc (de Wet et al., 1987) (Tse et al., 2002) and pG5GFP-hyg (Shioda et al., 2000) have been described. pRL-CMV was obtained from Promega Ltd.

Bait Expression Clones:—

The plasmids pM1-HRASG12V, pM1-LacZ (Tanaka & Rabbitts, 2003) and pBTM-ATF-2 (Portner-Taliana et al., 2000) have been described. pM1-ATF-2 was made by sub-cloning the Sma1-BamH1 fragment from pBTM-ATF-2 into the pM1 vector (Sadowski et al., 1992). For production of pM1-LexA, a LexA-DBD fragment was amplified from pBTM116 (Hollenberg et al., 1995) using BLEXAF2,5′-CGCGGATCCTGAAAGCGTTAACGGCCAGG-3′ and BAMLEXAR, 5′-CGCGGATCCAGCCAGTCGCCGTTGC-3′, and cloned into the BamH1 site of pM1.

Intrabody Expression Clones:—

The intrabody expression plasmids pEF-scFv33-VP16 (anti-RAS), pEF-scFvI21R33-VP16 (anti-RAS) (Tanaka & Rabbitts, 2003) and pEF-scFvR4-VP16 (anti-β-gal) (Martineau et al., 1998) (Tanaka et al., 2003) have been described. The clones pEF-33VH-VP16, pEF-I21R33VH-VP16, pEF-I21R33VH-C22S-VP16 and pEF-121R33VH-C92S-VP16 were made by PCR amplification of the VH domain fragments from the parental pEF-scFv-VP16 (using the oligonucleotides EFFP, 5′-TCTCAAGCCTCAGACAGTGGTTC-3′ and NotVHJR1′ 5′-CATGATGATGTGCGGCCGCTCCACCTGAGGAGACGGTGACC-3; the latter introduces a Not1 cloning site) and cloning into the Sfi1-Not1 sites of pEF-VP16 (Tanaka et al., 2003). The pEF-33VL-VP16 and pEF-I21R33VL-VP16 domain fragments were amplified from the parental pEF-scFv-VP16 using VLF1 5′-ATCATGCCATGGACATCGTGATGACCCAGTC-3′ (this introduces a Nco1 cloning site) plus VP162R, 5′-CAACATGTCCAGATCGAA-3′ and sub-cloned into the Nco1-Not1 sites of pEF-VP16 (Tanaka et al., 2003). pHEN2-scFv or IDab (for bacterial periplasmic expression) were made by cloning the Sfi1-Not1 fragments of the appropriate pEF-scFv-VP16 or pEF-IDab-VP16 into pHEN2 phagemid. The pZIPneoSV(X)-HRASG12V was made by cloning the coding sequence of HRASG12V mutant cDNA from pEXT-HRAS into the pZIPneoSV(X) vector (Cepko et al., 1984). The pEF-FLAG-memb-IDab clones were made by inserting the appropriate Sfi1-Not1 fragments of the pEF-IDab-VP16 clones into pEF-FLAG-Memb (Tanaka & Rabbitts, 2003).

All the above constructs were verified by sequencing.

Construction of Yeast IDab Libraries

The construction of the IDab libraries in the yeast prey expression vector pVP16* is described in detail elsewhere (Tanaka et al., 2003). Two IDab libraries were made (designated IDab library 1 and 2; Table 1). For library 1 preparation, the VH templates were from the previously described scFv, viz. scFvI21R33 or scFv625, which have intrabody VH consensus frameworks, of which the scFv625 has the canonical consensus (Tanaka et al., 2003; Tanaka & Rabbitts, 2003). The VH CDR2 and CDR3 regions of these scFvs were randomised by PCR mutagenesis (Hoogenboom & Winter, 1992; Tanaka et al., 2003) using NNM for codon redundancy in the CDRs (where N=A, G, C or T and M=T or G) and the products cloned into pVP16*, to encode VH-VP16 activation domain fusion proteins. This produced two diverse sets of clones with variability in the VH CDR2 and CDR3 regions. The total number of clones for the I21R33-derived library was approximately 2×10⁶ and of the scFv625 consensus-derived library was approximately 1.4×10⁶. These were combined to give a total of approximately 3.4×10⁶ clones (IDab library 1). For IDab library 2 preparation, the templates were the libraries described above. The CDR1 regions were randomised by mutagenesis (Hoogenboom & Winter, 1992; Tanaka et al., 2003) and cloned into pVP16*. This generated two diverse sets of clones with variability in the VH CDR1, CDR2 and CDR3 regions. The total number of clones obtained for the I21R33-derived library was approximately 3×10⁷ and approximately 2.2×10⁷ from the scFv625 consensus-derived library. These were combined to give a total of approximately 5.2×10⁷ clones (IDab library 2). The diversity of the libraries was estimated by determination of the total number of colony forming units and sequencing randomly picked clones to verify both the presence of VH segments (˜100% of clones had VH inserts) and the randornisation of CDRs. The latter showed that ˜57% of clones in the I21R33-derived library and ˜63% of clones in the scFv625 consensus-derived library had fully open reading frames in VH and VP16 fusions; the other clones had stop codons in either CDR1, CDR2 or CDR3, introduced during the randomisation process (for the I21R33-derived library ˜17%, ˜13% and ˜9% had stop codons in CDR1, CDR2 or CDR3 respectively; for the scFv625 consensus-derived library ˜5%, ˜26% and ˜5% had stop codons in CDR1, CDR2 or CDR3 respectively) and thus the diversity in each library could be estimated at ˜1.7×10⁷ and ˜1.4×10⁷ for I21R33-and scFv625 consensus-derived libraries respectively (i.e. combined library 2 of ˜3×10⁷).

Intracellular Antibody Capture (IAC) Screening of IDab Libraries

The screening of synthetic Dab libraries was performed according to the protocol of intracellular antibody capture (IAC) technology, as described (Tanaka & Rabbitts, 2003; Tse et al., 2002). A detailed protocol is available at http://www2.mrc-1mb.cam.ac.uk/PNAC/Rabbitts_T/group/index.html

In outline, 500%1 g of pBTM-antigen (bait) and 1 mg of the pVP16*-IDab library 1 or the pVP16*-IDab library 2 (preys) were co-transfected into S. cerevisiae L40. Positive clones were selected using the auxotrophic markers trp, leu and his. Positive clones were selected for his prototrophy and confirmed using β-galactosidase (β-gal) filter assays. For the selected clones, true positive clones were confirmed by re-testing histidine dependent growth and β-gal activation, using relevant and non-relevant baits. Ten double positive clones causing most rapid colour development in β-gal filter assays were selected and sequenced. More efficient selections can be achieved by first creating a yeast strain stably expressing the bait of interest (see also website indicated above).

Luciferase Assays and Western Blots

The luciferase procedure has been described previously (Tanaka & Rabbitts, 2003; Tse et al., 2002). scFv or IDab intrabodies were cloned into the pEF-VP16 expression vector and the antigen into the pM1 vector. COS7 cells (2×10⁵) were transiently co-transfected with 500 ng of pG5-Luc, 50 ng of pRL-CMV, 500 ng of pEF-scFv-VP16 or pEF-IDab-VP16 and 500 ng of pM1-antigen bait using 8 μl LipofectAMINE™ transfection reagent (Invitrogen), according to the Manufacture's instructions. 48 hours after transfection, the cells were washed, lysed and assayed using the Dual-Luciferase Reporter Assay System (Promega). Transfection efficiency was normalised to Renilla luciferase activity, which was obtained by co-transfection of pRL-CMV. The data represent two experiments, each performed in duplicate.

To confirm the expression of scFv-VP16 or IDab-VP16 fusion proteins, whole protein extracts were prepared by directly adding SDS-PAGE buffer to the transfected COS7 cell pellets. The lysates were analysed by SDS-PAGE, followed by Western detection using an anti-VP16 monoclonal antibody (145-, Santa-Cruz Biotechnology) as the primary antibody and an HRP-conjugated rabbit anti-mouse IgG antibody (Amersham-Pharmacia Biotech, APB) as the secondary antibody. The blots were visualised using an ECL detection kit (APB). Analysis of expression of scFv or IDab intrabodies in NIH3T3 cells (D4 line, a kind gift from Dr C. Marshall) was carried out as described (Tanaka & Rabbitts, 2003). D4 cells were transfected with pEF-FLAG-Memb-scFv or pEF-FLAG-Memb-IDab with or without pZIPneoSV(X)-HRASG12V. 48 hours after transfection, the cells were washed once with PBS, lysed in ice-cold lysis buffer (10 mM HEPES, pH 7.6, 250 mM NaCl, 5 mM EDTA, 0.5% NP-40, 1 μg/ml leupeptin, 1 μg/ml pepstatin A, 0.1 mg/ml aprotinin, imM phenylmethanesulfonyl fluoride) and the cells recovered by centrifugation at 4° C. The pellets (“insoluble” fraction) and the supernatants (“soluble” fraction) were analysed by SDS-PAGE, followed by Western detection using an anti-FLAG monoclonal antibody (M2. Sigma) as primary antibody.

Mammalian Two Hybrid Assays Using CD4 or GFP Reporter Cells

Chinese hamster ovary (CHO) cells were grown in minimal essential medium alpha (MEM-α, Invitrogen) supplemented with 10% foetal calf serum, penicillin and streptomycin. FACS analyses of the CHO-CD4 line (Fearon et al., 1992) were performed essentially as described before (Tse & Rabbitts, 2000). The CHO-GFP line was established by transfecting the pG5GFP-Hyg vector into CHO cells using LipofectAMINE™ and selecting transfected cells for 7 days in MEM-α containing 0.3 mg/ml hygromycin B (Sigma). The CHO-GFP stable clone 39a was chosen for further assays. For FACS assays, 3×10⁵ CHO-CD4 or CHO-GFP cells were seeded in 6-well plates twenty-four hours before transfection. 0.5 μg of pM1-antigen and 1 μg of pEF-scFv-VP16 or pEF-IDab-VP16 were co-transfected into the cells. Forty-eight hours after transfection, cells were washed, dissociated and re-suspended in PBS. For the CHO-CD4 assay, induction of cell surface CD4 expression was detected by using an anti-human CD4 antibody (RPA-T4, Pharmingen) and FITC-conjugated anti-mouse IgG antibody (Pharmingen). The fluorescence of CHO-CD4 or of CHO-GFP cells was measured with a FACSCalibur (Becton Dickinson) and the data were analysed by the CELLQuest software.

Purification of IDab Fragments In Vitro and BIAcore Affinity Measurement

For in vitro assays, scFvs and IDabs were expressed and isolated from the bacterial periplasm as previously described (Tanaka & Rabbitts, 2003). IDab fragments were cloned into the pHEN2 vector containing the pelB leader sequence with a His-tag and a myc-tag. IDabs were induced with 1 mM isopropyl-β,D-thiogalactopyranoside (IPTG) in 1 litre culture for 4 hours at 30° C. The cells were harvested and periplasmic fractions extracted in 10 ml of cold TES buffer (0.2 M Tris-HCl pH 7.5, 0.5 mM EDTA, 0.5 M sucrose). After dialysis against 2.5 litres of PBS, including 10 mM imidazole at 4° C., scFv and IDab fragments were purified using Ni-NTA agarose (QIAGEN), according to the Manufacture's instructions, concentrated using Centricon concentrators (YM-10, Amicon) and aliquots were stored at −70° C. Protein concentration was measured using a Bio-Rad Protein assay Kit according to the Manufacture's instructions. In vitro affinities of scFvs and IDabs were determined using surface plasmon resonance on a BIAcore 2000 instrument (Pharmacia Biosensor). The kinetic rate constants, kₒₙ and k_{off}, were calculated using the software supplied by the Manufacturer. Kd values were calculated from k_{off} and kₒₙ rate constants (Kd=k_{off}/kₒₙ). All measurements were performed in duplicate.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry, molecular biology and biotechnology or related fields are intended to be within the scope of the following claims.
Adjei, A. A. (2001) Blocking oncogenic Ras signaling for cancer therapy. J Natl Cancer Inst, 93, 1062-74.
Biocca, S., Pierandrei-Amaldi, P., Campioni, N. and Cattaneo, A. (1994) Intracellular immunization with cytosolic recombinant antibodies. Biotechnology, 12, 396-399.
Biocca, S., Pierandrei-Amaldi, P. and Cattaneo, A. (1993) Intracellular expression of anti-p21ras single chain Fv fragments inhibits meiotic maturation of xenopus oocytes. Biochem Biophys Res Commun, 197, 422-7.
Biocca, S., Ruberti, F., Tafani, M., Pierandrei-Amaldi, P. and Cattaneo, A. (1995) Redox state of single chain Fv fragments targeted to the endoplasmic reticulum, cytosol and mitochondria. Biotechnology (N Y), 13, 1110-5.
Bird, R. E., Hardman, K. D., Jacobson, J. W., Johnson, S., Kaufman, B. M., Lee, S.-M., Lee, T., Pope, S. H., Riordan, G. S. and Whitlow, M. (1988) Single-chain antigen-binding proteins. Science, 242, 423-426.
Cattaneo, A. and Biocca, S. (1999a) The selection of intracellular antibodies. Trends Biotech
Cattaneo, A. and Biocca, S. (1999b) The selection of intracellular antibodies. Trends Biotechnol, 17, 115-21.
Cepko, C. L., Roberts, B. E. and Mulligan, R. C. (1984) Construction and applications of a highly transmissible murine retrovirus shuttle vector. Cell, 37, 1053-62.
Cohen, P. A. (2002) Intrabodies. Targeting scFv expression to eukaryotic intracellular compartments. Methods Mol. Biol., 178, 367-378.
de Wet, J. R., Wood, K. V., DeLuca, M., Helinski, D. R. and Subramani, S. (1987) Firefly luciferase gene: structure and expression in mammalian cells. Mol Cell Biol, 7, 725-37.
de Wildt, R. M., Mundy, C. R., Gorick, B. D. and Tomlinson, I. M. (2000) Antibody arrays for high-throughput screening of antibody-antigen interactions. Nat Biotechnol, 18, 989-94.
Herrmann, C., Horn, G., Spaargaren, M. and Wittinghofer, A. (1996) Differential interaction of the ras family GTP-binding proteins H-Ras, Rap1A, and R-Ras with the putative effector molecules Raf kinase and Ral-guanine nucleotide exchange factor. J Biol Chem, 271, 6794-800.
Hollenberg, S. M., Stemglanz, R., Cheng, P. F. and Weintraub, H. (1995) Identification of a new family of tissue-specific basic helix-loop-helix proteins with a two-hybrid system. Molecular and Cellular Biology, 15, 3813-3822.
Huston, J. S., Levinson, D., Mudgett-Hunter, M., Tai, M.-S., Novoty, J., Margolies, M. N., Ridge, R. J., Bruccoleri, R. E., Haber, E., Crea, R. and Oppermann, H. (1988) Protein engineering of antibody binding sites: Recovery of specific activity in an anti-digoxin single-chain Fv analogue produced in Escherichia coli. Proc. Natl. Acad. Sci. USA, 85, 5879-5883.
Jung, S., Spinelli, S., Schimmele, B., Honegger, A., Pugliese, L., Cambillau, C. and Pluckthun, A. (2001) The importance of framework residues H6, H7 and H10 in antibody heavy chains: experimental evidence for a new structural subclassification of antibody V(H) domains. J Mol Biol, 309, 701-16.
Kabat, E. A., Wu, T. T., Perry, H. M., Gottesman, K. S. and Foeller, C. (1991) Sequences of proteins of immunological interest. National Institutes of Health, Bethesda.
Lefranc, M.-P. and Lefranc, G. (2001) The Immunoglobulin Factsbook. Academic Press, London.
Lowy, D. R. and Willumsen, B. M. (1993) Function and regulation of ras. Annu Rev Biochem, 62, 851-91.
Marasco, W. A., Haseltine, W. A. and Chen, S. (1993) Design, intracellular expression, and activity of a human anti-human immunodeficient virus type 1 gp120 single-chain antibody. Proc. Natl. Acad. Sci. USA, 90, 7899-7893.
Martineau, P., Jones, P. and Winter, G. (1998) Expression of an antibody fragment at high levels in the bacterial cytoplasm. J Mol Biol, 280, 117-127.
Mizushima, S. and Nagata, S. (1990) pEF-BOS, a powerful mammalian expression vector. Nucl. Acids Res., 18, 5322.
Morea, V., Tramontano, A., Rustici, M., Chothia, C. and Lesk, A. M. (1998) Conformations of the third hypervariable region in the VH domain of immunoglobulins. J Mol Biol, 275, 269-94.
Pacold, M. E., Suire, S., Perisic, O., Lara-Gonzalez, S., Davis, C. T., Walker, E. H., Hawkins, P. T., Stephens, L., Eccleston, J. F. and Williams, R. L. (2000) Crystal structure and functional analysis of Ras binding to its effector phosphoinositide 3-kinase gamma. Cell, 103, 93143.
Proba, K., Worn, A., Honegger, A. and Pluckthun, A. (1998) Antibody scFv fragments without disulfide bonds made by molecular evolution. J Mol Biol, 275, 245-53.
Rabbitts, T. H. (1994) Chromosomal translocations in human cancer. Nature, 372, 143-149.
Rabbitts, T. H. and Stocks, M. R. (2002) Chromosomal translocation products engender novel intracellular therapeutic technologies. submitted.
Rondon, I. J. and Marasco, W. A. (1997) Intracellular antibodies (intrabodies) for gene therapy of infectious diseases. Annu Rev Microbiol, 51, 257-83.
Sadowski, I., Bell, B., Broad, P. and Hollis, M. (1992) GAL4 fusion vectors for expression in yeast or mammalian cells. Gene, 118, 137-141.
Sheets, M. D., Amersdorfer, P., Finnern, R., Sargent, P., Lindqvist, E., Schier, R., Hemingsen, G., Wong, C., Gerhart, J. C. and Marks, J. D. (1998) Efficient construction of a large nonimmune phage antibody library: the production of high-affinity human single-chain antibodies to protein antigens. Proc. Natl. Acad Sci. USA, 95, 6157-6162.
Tanaka, T., Chung, G. T. Y., Forster, A., Lobato, M. N. and Rabbitts, T. H. (2002) De novo synthesis of antibody gene fragments and diversification by footprint mutagenesis. in prep.
Tavladoraki, P., Benvenuto, E., Trinca, S., De Martinis, D., Cattaneo, A. and Galeffi, P. (1993) Transgenic plants expressing a functional single-chain Fv antibody are specifically protected from virus attack. Nature, 366, 469-472.
Triezenberg, S. J., Kingsbury, R. C. and McKnight, S. L. (1988) Functional dissection of VP16, the trans-activator of herpes simplex virus immediate early gene expression. Genes Dev, 2, 718-29.
Tse, E., Chung, G. and Rabbitts, T. H. (2000) Protocols for isolating antigen-specific intracellular antibody fragments as sinlge chain Fv for use in mammalian cells. In Walker, J. and Turksen, K. (eds.), Methods in Molecular Biology and Medicine. Humana Press, Totawa, Vol. in press.
Tse, E., Lobato, M. N., Forster, A., Tanaka, T., Chung, T. Y. G. and Rabbitts, T. H. (2002) Intracellular antibody capture technology: application to selection of single chain Fv recognising the BCR-ABL oncogenic protein. J. Mol. Biol., 317, 85-94.
Tse, E. and Rabbitts, T. H. (2000) Intracellular antibody-caspase mediated cell killing: a novel approach for application in cancer therapy. Proc. Natl. Acad. Sci. USA, 97, 12266-12271.
Visintin, M., Settanni, G., Maritan, A., Graziosi, S., Marks, J. D. and Cattaneo, A. (2002) The intracellular antibody capture technology (IACT): towards a consensus sequence for intracellular antibodies. J. Mol. Biol, 317, 73-83.
Worn, A. and Pluckthun, A. (1998a) An intrinsically stable antibody ScFv fragment can tolerate the loss of both disulfide bonds and fold correctly. Febs Lett., 427, 357-361.

Worn, A. and Pluckthun, A. (1998b) Mutual stabilization of VL and VH in single-chain antibody fragments, investigated with mutants engineered for stability. Biochemistry, 37, 13120-7.

TABLE 1

| | | | No. of clones | |
|---|---|---|---|---|
| Bait | | No. clones | HIS - | β-gal |
| (antigen) | Library | screened | growth | positive |
| HRASG12V | IDab library 1 | 7.86 × 10⁷ | 454 | 374 |
| | IDab library 2 | 1.65 × 10⁸ | 510 | 488 |
| ATF-2 | IDab library 1 | 1.18 × 10⁷ | 314 | 277 |

TABLE 2

| scFv/IDab | Kₒₙ(M⁻¹s⁻¹) | K_{off}(s⁻¹) |
|---|---|---|
| scFv33 | 1.76 ± 1.41 × 10⁵ | 1.13 ± 0.16 × 10⁻³ |
| scFvl21R33VHl21VL | 4.78 ± 0.95 × 10⁴ | 8.65 ± 0.78 × 10⁻⁴ |
| IDab 33 | 1.25 ± 0.12 × 10⁴ | 1.44 ± 0.68 × 10⁻² |
| IDab anti-RAS #3 | 5.66 ± 0.18 × 10³ | 1.04 ± 0.01 × 10⁻³ |
| IDab anti-RAS #10 | 2.32 ± 1.17 × 10⁴ | 2.54 ± 0.34 × 10⁻³ |
| IDab anti-RAS #12 | 2.73 ± 1.12 × 10⁴ | 7.05 ± 2.28 × 10⁻⁴ |

1. An antibody molecule capable of specifically binding to activated RAS within an intracellular environment wherein the antibody comprises a single variable domain type only and such variable domain comprises any of the amino acid sequences selected from the groups consisting of: (a) in the case of VH: Con, J4S, 33, I21R33, I21R33VHI21VL, Con 33 and I21R33(VHC22S;C92S) as depicted in FIG. 3 and designated SEQ ID NO:1, SEQ ID NO: 2, SEQ ID NO: 3, SEQ ID NO: 7, SEQ ID NO: 8, SEQ ID NO: 9, SEQ ID NO: 10 respectively, or any of the sequences listed above 10 in which one or more of residues 22 and 92 are not cysteine residues SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29 as depicted in FIG. 3; and (b) in the case of VL: Con, J48, 33, I21R33, I21R33VHI21VL, Con 33 and I21R33(VHC22S;C92S) as depicted in FIG. 3 and designated SEQ ID NO: 1, SEQ ID NO: 12, SEQ ID NO: 13, SEQ ID NO: 17, SEQ ID NO: 18, SEQ ID NO: 19, SEQ 20 respectively.

2. A antibody according to claim 1 which comprises one or more heavy chain variable domains and not one or more light chain variable domains.

3. An antibody according to claim 1 which comprises one or more ligh chain variable domains and not one or more heavy chain variable domains.

4. An antibody molecule capable of specifically binding to activated RAS within an intracellular environment wherein the antibody comprises a heavy chain variable 25 domain and a light chain variable domain wherein the heavy chain variable domain and the light chain variable domain of the antibody comprise any of the amino acid sequences selected from the group consisting of: Con, J48, 33, I21R33, I21R33VHI21VL, Con 33 and I21R33(VHC22S,C92S) as depicted in FIG. 3 and designated SEQ ID NO: 1, SEQ ID NO: 2, SEQ ID NO:3, SEQ ID NO: 7, SEQ ID NO: 8, SEQ ID NO: 9 and SEQ ID NO: 10 in the case of 30 variable heavy chain domains or any of the sequences listed above in which one or more of residues 22 and 92 (according to Kabat numbering) are not cysteine residues, and the corresponding light chain domains as depicted in FIG. 3.

5. An antibody molecule for functionally inactivating activated RAS within an intracellular environment wherein the antibody comprises a single variable domain type only and such variable domain comprises any of the amino acid sequences 5 selected from the groups consisting of: (a) in the case of VH: Con, J48, 33, I21R33, I21R33VHI21VL, Con 33 and I21R33(VHC22S;C92S) as depicted in FIG. 3 and designated SEQ 1, SEQ ID NO.: 2, SEQ ID NO: 3, SEQ ID NO: 7, SEQ ID NO: 8, SEQ ID NO: 9, SEQ ID NO: 10, respectively; or any of the sequences listed above in which one or more of residues 22 and 92 are not cysteine residues; SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29 as depicted in FIG. 3; and (b) in the case of VL: Con, J48, 33, 121R33, I21R33N7HI21VL, Con 33 and I21R33(VHC22S;C92S) as depicted in FIG. 3 and designated SEQ ID NO: 11, SEQ ID NO: 12, SEQ ID NO: 13, SEQ17, SEQ ID NO:18, SEQ ID NO: 19, SEQ ID NO: 20 respectively.

6. An antibody molecule for functionally inactivating activated RAS within an intracellular environment wherein the antibody comprises a heavy chain variable domain and a light chain variable domain wherein the heavy chain variable domain and the light chain variable domain of the antibody comprise any of the amino acid sequences selected from the group consisting of: Con, J48, 33, I21R33, I21R33VHI21VL, Con 33 and I21R33(VHC22S;C92S) as depicted in FIG. 3 and designated SEQ ID NO: 1, SEQ ID NO: 2, SEQ ID NO:3, SEQ ID NO: 7, SEQ ID NO: 8, SEQ ID NO: 9 and SEQ ID NO: 10 respectively in the case of variable heavy chain domains or any of the sequences listed above in which one or more of residues 22 and 92 (according to Rabat numbering) are not 25 cysteine residues and the corresponding light chain domains as depicted in FIG. 3.

7. A single variable domain type anti-activated RAS intracellularly binding antibody comprising a set of variable heavy or light chain domain (CDRs selected from the group shown in FIG. 3 and depicted SEQ ID NO: 1a, b and c; SEQ ID NO: 2 a, b and c; SEQ ID NO: a, b, and c; SEQ ID NO: 11a, b and c; SEQ ID NO: 12 a, b and c; and SEQ ID NO: 3, a, b, c; SEQ ID NO: 21 a, band c; SEQ ID NO: 22 a, b and c; SEQ ID NO: 23 a, b and c; SEQ ID NO: 24 a, b and c; SEQ ID NO: 25 a, b and c; SEQ ID NO: 26 a, b and c; SEQ ID NO: 27 a, b and c; SEQ ID NO: 28a, band c; SEQ ID NO: 29 a, b and c.

8. An anti-activated RAS intracellularly binding antibody comprising at least one 5 light and at least one heavy chain domain wherein the antibody comprises those variable heavy chain domain CDRs selected from the group shown in FIG. 3 and depicted SEQ ID NO: 1a, b and c; SEQ ID NO: 2 a, b and c; and SEQ ID NO: 3, a, b, c; and the corresponding light chain domain CDRs selected from the group shown in FIG. 3 and depicted SEQ ID NO: 11a, b and c; SEQ ID NO: 12 a, b and c; and SEQ ID NO: 13, a, b, c.

9. Those variable domain CDRs selected from those amino acid sequences shown in FIG. 3 and depicted SEQ ID NO: 1a, b and c; SEQ ID NO: 2 a, b and c; SEQ ID NO: 3, a, b, c; SEQ ID NO: 1 a, b, c; SEQ ID NO: 12 a, b, c, SEQ ID NO: 13 a, b, c; SEQ ID NO: 21 a, b and c; SEQ ID NO: 22 a, b and c; SEQ ID NO: 23 a, b and c; SEQ ID NO: 24 a, b and c, SEQ ID NO: 5 a, b and c; SEQ ID NO: 26 a, b and c; SEQ ID NO: 27 a, b and c; SEQ ID NO: 28 a, b and c; SEQ ID NO: 29 a, b and c and which when attached to their respective heavy or light chain variable domain framework regions amino acid sequences to generate an intracellularly functional antibody, confer upon the resultant antibody the ability to selectively bind to activated RAS within an intracellular environment.

10. A nucleic acid construct encoding an antibody molecule according to claim 1 and/or any a CDR sequence according to claim 9.

11. A vector comprising a nucleic acid construct according to claim 10.

12. A host cell transformed with a vector according to claim 11.

13. A composition comprising a molecule selected from the group consisting of an antibody molecule according to claim 1, 4, 5, 6, 7 or 8, CDRs according to claim 9, and a nucleic acid construct according to claim 10 and a pharmaceutically acceptable carrier, diluent or exipient.

14. A method for generating an antibody molecule which is capable of specifically binding to activated RAS and/or functionally inactivating activated RAS within an intracellular environment comprising the step of synthesising the antibody from a variable chain domain comprising any of those amino acids sequences selected from the group shown in FIG. 3 and designated for VH: SEQ ID NO: 1, 2, 3, 7, 8, 9, 10 or from any of those listed VH sequences in which one or more of residues 22 and 92 (according to Kabat numbering) are not cysteine residues; and/or synthesising the antibody from a variable chain domain comprising any of those amino acids selected from the group shown in FIG. 3 and designated SEQ ID NO: 11, 12, 13, 17, 18, 19 and 20 and depicted Con, J48, 33, I21R33, I21R33VHI21VL, Con33, I21R33 (VHC22S, C92S) respectively.

15. An antibody obtained by the method of claim 14.

16. The method of inhibiting the functional activity of activated RAS within an intracellular environment, the method comprising contacting an antibody molecule comprising a light and/or heavy chain variable domain comprising any of those amino acids sequences selected from the group shown in FIG. 3 and designated for VH: SEQ ID NO: 1, 2, 3, 7, 8, 9, 10, or from any of those listed VH sequences in which one or more of residues 22 and 92 (according to Kabat numbering) are not cysteine residues or any of those amino acid sequences selected from the group shown in FIG. 3 and designated in the case of VH: SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28 and SEQ ID NO: 29; and/or a variable light chain domain comprising any of those amino acids selected from the group shown in FIG. 3 and designated SEQ ID NO: 11, 12, 13, 17, 18, 19 and 20 and depicted Con, J48, 33, I21R33, I21R33VHI21VL, Con33, I21R33 (VHC22S, C92S) respectively with activated RAS and/or inhibiting the in vivo functional activity of activated RAS within an intracellular environment.

17. The method according to claim 16 wherein said antibody molecule is a single variable domain type antibody.

18. The method of claim 17 wherein the variable domain is a heavy chain variable domain.

19. The method of claim 17 wherein the variable domain is a light chain variable domain.

20. The method of claim 16 wherein the antibody comprises both light and heavy chain variable domains.

21. A method for the treatment of RAS associated cancer in a patient comprising the steps of administering to the patient in need of such treatment a therapeutically effective amount of one or more antibody molecule/s comprising a light and/or heavy chain variable domain comprising any of those amino acids sequences selected from 15 the group shown in FIG. 3 and designated for VH: SEQ ID NO: 1, 2, 3, 7, 8, 9, 10; or from any of those listed VH sequences in which one or more of residues 22 and 92 (according to Rabat numbering) are not cysteine residues, any of those amino acids sequences selected from the group shown in FIG. 3 and designated for VH: SEQ ID NO: 21, 22, 23, 24, 25, 26, 27, 28 and SEQ ID NO: 29; and/or a variable chain domain comprising 20 any of those amino acids selected from the group shown in FIG. 3 and designated SEQ ID NO: 11, 12, 13, 17, 18, 19 and 20 and depicted Con, J48, 33, I21R33, I21R33VHI21VL, Con33, I21R33 (VHC22S, C92S) respectively.

22. The method of claim 21 wherein the antibody is a single variable domain type antibody.

23. The method of claim 22 wherein the antibody is a heavy chain variable domain only antibody.

24. The method of claim 22 wherein the antibody is a light chain variable domain type antibody.

25. The method of claim 21 wherein the antibody comprises both light and heavy chain variable domains.
